(19) United States
(12) Patent Application Publication
(10) Pub. No.: US 2015/0110865 A1
(43) Pub. Date: Apr. 23, 2015

(54) CNS STIMULANT AND OPIOID RECEPTOR ANTAGONIST COMBINATION AS A NON-ADDICTIVE, NON-AVERSIVE AND SYNERGISTIC ANTI-OBESITY TREATMENT

(71) Applicants: FLORIDA STATE UNIVERSITY RESEARCH FOUNDATION, Tallahasse, FL (US); THE GENERAL HOSPITAL CORPORATION, Boston, MA (US)

(72) Inventors: PRADEEP G. BHIDE, TALLAHASSEE, FL (US); JINMIN ZHU, TALLAHASSEE, FL (US); JOSEPH BIEDERMAN, BROOKLINE, MA (US); THOMAS J. SPENCER, CARLISLE, MA (US)

(21) Appl. No.: 14/519,222

(22) Filed: Oct. 21, 2014

Related U.S. Application Data
(60) Provisional application No. 61/893,571, filed on Oct. 21, 2013.

(51) Int. Cl. A61K 31/485; A61K 31/4458

(52) U.S. Cl. CPC A61K 31/485; A61K 31/4458
USPC 424/451; 514/282

(57) ABSTRACT

Combinations comprise a therapeutically effective amount of one or more stimulants and and/or pharmaceutically acceptable analogs, salts, or hydrates of the one or more stimulants, and one or more non-selective opioid receptor antagonists, and/or pharmaceutically acceptable analogs, salts or hydrates of the one or more non-selective opioid receptor antagonists. These combinations may be used for treating obesity via administration to a subject having a need thereof.

### CROSS-REFERENCE TO RELATED APPLICATIONS

This application claims benefit of priority to U.S. Provisional Patent Application No. 61/893,571 to Bhide et al., entitled “CNS Stimulant and Opioid Receptor Antagonist Combination As a Non-Addictive, Non-Aversive and Synergistic Anti-Obesity Treatment,” filed Oct. 21, 2013. The entire contents and disclosure of this patent application are incorporated herein by reference in its entirety.

This application makes reference to the following U.S. patents and U.S. patent applications: U.S. Provisional Patent Application No. 61/716,769, entitled “Novel Class of Non-stimulant Treatment for ADHD and Related Disorders,” filed Oct. 22, 2012; U.S. patent application Ser. No. 14/027,676, entitled “Novel Class of Non-stimulant Treatment for ADHD and Related Disorders,” filed Sep. 16, 2013; U.S. Provisional Patent Application No. 61/877,147, entitled “Selective Dopamine D4 Receptor Agonists for Treatment of Working Memory Deficits,” filed Sep. 13, 2013; and U.S. Provisional Patent Application No. 61/893,571, entitled “CNS Stimulant and Opioid Receptor Antagonist Combination as a Non-Addictive, Non-Aversive and Synergistic Anti-Obesity Treatment,” filed Oct. 21, 2013. The entire contents and disclosure of these patent applications are incorporated herein by reference in their entirety.

### BACKGROUND

1. Field of the Invention

The present invention relates generally to the treatment of obesity.

2. Related Art

Obesity is a complex disorder involving an excessive amount of body fat. It increases the risk of various diseases and health problems such as heart disease, diabetes, high blood pressure, obstructive sleep apnea, certain types of cancer, and osteoarthritis, etc.

### SUMMARY

According to a first broad aspect, the present invention provides a method for treating obesity comprising administering to a subject having a need thereof a combination comprising two or more compounds, wherein the two or more compounds comprise: a therapeutically effective amount of naltrexone and/or pharmaceutically acceptable analogs, salts, or hydrates of naltrexone; and a therapeutically effective amount of methylphenidate and/or pharmaceutically acceptable analogs, salts, or hydrates of methylphenidate.

According to a second broad aspect, the present invention provides a method for treating obesity comprising administering a combination to a subject having a need thereof, wherein the combination comprises two or more compounds, and wherein the two or more compounds comprise: a therapeutically effective amount of one or more non-selective opioid receptor antagonists and/or pharmaceutically acceptable analogs, salts, or hydrates of the one or more non-selective opioid receptor antagonists; and a therapeutically effective amount of one or more CNS stimulants and/or pharmaceutically acceptable analogs, salts, or hydrates of the one or more CNS stimulants.

According to a third broad aspect, the present invention provides a product for treating obesity. The product comprises at least one dosage of a combination, wherein the combination comprises two or more compounds, and wherein the two or more compounds comprise: a therapeutically effective amount of naltrexone and/or pharmaceutically acceptable analogs, salts, or hydrates of naltrexone; and a therapeutically effective amount of methylphenidate and/or pharmaceutically acceptable analogs, salts, or hydrates of methylphenidate.

According to a fourth broad aspect, the present invention provides a product for treating obesity. The product comprises at least one dosage of a combination for treating obesity, wherein the combination comprises a therapeutically effective amount of two or more compounds, and wherein the two or more compounds comprise: one or more non-selective opioid receptor antagonists and/or pharmaceutically acceptable analogs, salts or hydrates of the one or more non-selective opioid receptor antagonists; and one or more CNS stimulants and/or pharmaceutically acceptable analogs, salts, or hydrates of the one or more CNS stimulants.

According to a fifth broad aspect, the present invention provides a treatment delivery apparatus comprising a treatment carrier device and at least one dosage of a combination contained in the treatment carrier device for treating obesity, wherein the combination comprises: a therapeutically effective amount of one or more non-selective opioid receptor antagonists and/or pharmaceutically acceptable analogs, salts or hydrates of the one or more non-selective opioid receptor antagonists; and a therapeutically effective amount of one or more CNS stimulants and/or pharmaceutically acceptable analogs, salts, or hydrates of the one or more CNS stimulants.

According to a sixth broad aspect, the present invention provides a treatment delivery apparatus comprising a transdermal patch, wherein the transdermal patch comprises an adhesive patch for placing the transdermal patch on a skin of a subject and one or more active layer embedded in the adhesive patch, wherein the one or more active layer comprise a combination comprising a therapeutically effective amount of two or more compounds for treating obesity, and wherein the two or more compounds comprise: one or more non-selective opioid receptor antagonists and/or pharmaceutically acceptable analogs, salts or hydrates of the one or more non-selective opioid receptor antagonists; and one or more CNS stimulants and/or pharmaceutically acceptable analogs, salts, or hydrates of the one or more CNS stimulants.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features of the invention.

FIG. 1 is a graph illustrating the effect of high-dose methylphenidate (MPH) on inducing conditioned place preference (CPP) in a mouse model according to an exemplary embodiment of the present invention;

FIG. 2 is a graph showing the effect of high-dose MPH treatment on the conditioned place preference (CPP) scores in a mouse model according to an exemplary embodiment of the present invention;

FIG. 3 is a graph showing that [³⁵S]GTPγS binding in membrane preparations from the caudate-putamen is increased by a selective μ-opioid receptor (MOPR) agonist DAMGO in a concentration-dependent manner according to an exemplary embodiment of the present invention;

FIG. 4 is a graph showing the effect of high-dose MPH on μ-opioid receptor (MOPR) activity according to an exemplary embodiment of the present invention;

FIG. 5 is a graph illustrating the effect of naltrexone on reducing MPH-induced conditioned place preference (CPP) according to an exemplary embodiment of the present invention;

FIG. 6 is a graph illustrating the effect of naltrexone on reducing MPH-induced CPP score according to an exemplary embodiment of the present invention;

FIG. 7 is a graph illustrating the effect of naltrexone on reducing MPH-induced MOPR activation according to an exemplary embodiment of the present invention;

FIG. 8 is an image showing a combination formulated in the dosage form of a softgel according to an exemplary embodiment of the present invention;

FIG. 9 is an image showing a combination formulated in the dosage form of a hard capsule according to an exemplary embodiment of the present invention;

FIG. 10 is an image showing a combination formulated in the dosage form of a hard capsule with compounds coated differently according to an exemplary embodiment of the present invention;

FIG. 11 is an image showing a combination formulated in the dosage form of a tablet according to an exemplary embodiment of the present invention;

FIG. 12 is an image showing a combination formulated in the dosage form of chewable tablet according to an exemplary embodiment of the present invention;

FIG. 13 is an image showing a combination formulated in the dosage form of caplet according to an exemplary embodiment of the present invention;

FIG. 14 illustrates a dosage form of a caplet comprising a core encased in a shell according to an exemplary embodiment of the present invention;

FIG. 15 illustrates a treatment delivery apparatus comprising a transdermal patch for delivering the combination disclosed herein into a subject's body according to an exemplary embodiment of the present invention;

FIG. 16 illustrates a transdermal patch comprising a combination disclosed herein embedded within the transdermal patch according to an exemplary embodiment of the present invention.

### DETAILED DESCRIPTION OF THE PREFERRED EMBODIMENTS

### Definitions

Where the definition of terms departs from the commonly used meaning of the term, applicant intends to utilize the definitions provided below, unless specifically indicated.

For purposes of the present invention, it should be noted that the singular forms, “a,” “an” and “the” include reference to the plural unless the context as herein presented clearly indicates otherwise.

For purposes of the present invention, the term “additive” refers to a substance added to another in relatively small amounts to effect a desired change in properties. In foods, an additive may be any of various chemical substances added to produce desirable effects. Additives include such substances may be artificial or natural colorings and flavorings; stabilizers, emulsifiers, and thickeners; preservatives and humectants (moisture-retainers); and supplementary nutrients, etc. For example, an additive in drinking water could be sugar, saccharin, salt, etc.

For purposes of the present invention, the term “administering in conjunction with” refers to administering respective formulations sequentially, separately and/or simultaneously, over the course of treatment of the relevant condition, which condition may be acute or chronic. In some embodiments, the two formulations are administered (possibly repeatedly) sufficiently closely in time for there to be a beneficial effect for the subject, that is greater, over the course of the treatment of the relevant condition, than if either of the two formulations are administered (optionally repeatedly) alone, in the absence of the other formulation, over the same course of treatment. Determination of whether a combination provides a greater beneficial effect in respect off and over the course of treatment of, a particular condition, will depend upon the condition to be treated or prevented, but may be achieved routinely by the person skilled in the art. Thus, the term “in conjunction with” includes that one or other of the two formulations may be administered (optionally repeatedly) prior to, after, and/or at the same time as, administration with the other component. When used in this context, the terms “administered simultaneously” and “administered at the same time as” include the possibility that separate doses are administered, for example, within 48 hours, 24 hours, 18 hours, 12 hours, 6 hours, 3 hours, 2 hours, 1 hour, or 30 minutes of each other. The typical daily dose of the active ingredients varies within a broad range and may depend on various factors such as the relevant indication, the route of administration, the age, weight and sex of the subject and may be determined by a physician.

For purposes of the present invention, the term “agonism” refers to an action of a substance which ultimately produces the same effects in a body as another substance, as if they are both agonists of a same receptor.

For purposes of the present invention, the term “agonist” refers to a compound that binds to a receptor and activates the receptor to produce a biological response.

For purposes of the present invention, the term “analogue” and the term “analog” refer to one of a group of chemical compounds that share structural and/or functional similarities but are different in respect to elemental composition. A structural analog is a compound having a structure similar to that of another one, but differing from it in respect of one or more components, such as one or more atoms, functional groups, or substructures, etc. Functional analogs are compounds that has similar physical, chemical, biochemical, or pharmacological properties. Functional analogs are not necessarily also structural analogs with a similar chemical structure.

For purposes of the present invention, the term “antagonist” refers to a compound that binds to a receptor and blocks or disrupts the action of an agonist at the receptor.

For purposes of the present invention, the term “Attention Deficit/Hyperactivity Disorder (ADHD)” refers to conditions such as ADHD, ADHD NOS, Hyperkinetic Disorder, Attention Deficit Disorder with and without Hyperactivity, and others, as defined by DSM III, DSM III-R, DSM IV, DSM IV-TR, DSM V, future DSM definitions, ICD 8, ICD 9, ICD 10 and future versions of ICD as well as similar definitions of ADHD. For purposes of the present invention, the term “ADHD” includes both full and subthreshold conditions where there are not sufficient ADHD symptoms to meet full diagnostic criteria, late onset of ADHD symptoms and ADHD symptoms that occur in the context of comorbid disorders, after head trauma or due to unknown etiology.

For purposes of the present invention, the term “binge eating disorder” refers to a disorder of compulsive overeating in which a subject consumes huge amounts of food while feeling out of control and powerless to stop. A binge eating episode in a person having binge eating disorder typically lasts around two hours. Some people binge on and off all day long. Binge eaters often eat even when they are not hungry and continue eating long after they are full.

For purposes of the present invention, the term “body mass index (BMI)” refers to an individual's body mass divided by the square of their height—with the value universally being given in units of kg/m². BMI is used in a wide variety of contexts as a simple method to assess how much an individual's body weight departs from what is normal or desirable for a person of his or her height.

For purposes of the present invention, the term “capsule” refers to a gelatinous envelope enclosing an active substance. Capsules may be soft-shelled capsules (softgels) or hard-shelled capsules. Capsules can be designed to remain intact for some hours after ingestion in order to delay absorption. They may also contain a mixture of slow- and fast-release particles to produce rapid and sustained absorption in the same dose.

For purposes of the present invention, the term “carrier” refers to relatively nontoxic chemical compounds or agents that facilitate the incorporation of a compound of interest such as naltrexone

For purposes of the present invention, the term “CNS stimulant” or the term “stimulant” refers to a compound that induces temporary improvements in either mental or physical functions or both. A CNS stimulant may temporarily increase alertness and energy. CNS Stimulants in embodiments disclosed herein include stimulant-like compounds and members in a class of drug that blocks the re-uptake of multiple neurotransmitters, such as dopamine, serotonin, noradrenaline, etc.

For purposes of the present invention, the term “co-administration” refers to administration of two or more compositions or compounds to a single subject. Each of the two or more compositions may be administered by the same or different route of administration, at the same time or different time. Co-administration of first therapeutically effective compound and a second therapeutically effective compound, which for example, may be dissolved or intermixed in the same pharmaceutically acceptable carrier.

For purposes of the present invention, the term “combination” refers to both a “fixed-dose combination” or a “co-packaged drug products.” A “fixed-dose combination” or a “fixed combination” is a formulation that includes two or more active pharmaceutical ingredients, e.g., medicaments, compounds, physically combined in a single dosage form. In another words, medicaments or compounds may be dissolved or intermixed in a same pharmaceutically acceptable carrier. The form of a single dosage can be, but is not limited to, a tablet, a softgel, a capsule, a hard capsule, a caplet, a chewable tablet, a gummy, an injection fluid, a transdermal patch, etc. A “combination product” refers to a product that combines drugs, devices, and/or biological products. Sometimes, a combination product may be a polypill or a combo pill in the dosage form such as a tablet, a capsule, etc. Sometimes, a “combination product” may a “non-fixed combination” or a “co-packaged drug product” in which two or more separate dosage forms packaged together in a single package or as a unit. Drug, device, or biological product may be packaged separately according to specific needs such as proposed labeling. The contents of a “non-fixed combination” may be administered to a subject simultaneously, concurrently, or sequentially at different time intervals or with no specific intervening time limits, wherein such administration provides effective levels of the medicaments or compounds in the body of the subject. A “combination administration” includes co-administration of various compounds in therapeutically effective amount, wherein the various compounds may be in a “fixed-dose combination” or in a “non-fixed combination.” A “concurrent administration” includes the administration of various compounds separately at the same time or sequentially in any order at different points in time to provide an effect suitable for the treatment. Therapy being either concomitant or sequential may be dependent on the characteristics of the other medicaments or compounds used, characteristics like onset and duration of action, mechanism of action, rate of absorption from the gastrointestinal tract, plasma levels, clearance, etc.

For purposes of the present invention, the term “comprising”, the term “having”, the term “including,” and variations of these words are intended to be open-ended and mean that there may be additional elements other than the listed elements.

For purposes of the present invention, the term “controlled release” refers to time dependent release. Timed release has several distinct variants such as sustained release where prolonged release is intended, pulse release, delayed release, etc. Time dependent release may be in oral dose formulations such as pills, capsules, gels, and may also in formulations such as implants, and devices, and transdermal patches.

For purposes of the present invention, the term “coating” or the term “enteric coating” refers to a polymer barrier applied on oral medication.

For purposes of the present invention, the term “daily dose” refers to the total dosage amount administered to an individual in a single 24-hour day.

For purposes of the present, the term “delayed release” refers to oral medicines that do not immediately disintegrate and release the active ingredient(s) into the body. For example, an enteric coated oral medication dissolves in the intestines rather than the stomach.

For purposes of the present invention, the term “dietary supplement” refers to a product taken by mouth that contains a “dietary ingredient” intended to supplement the diet. The “dietary ingredients” in these products may include: vitamins, minerals, herbs or other botanicals, amino acids, and substances such as enzymes and metabolites. Dietary supplements may also be extracts or concentrates, and may be found in many dosage forms such as tablets, hard capsules, softgels, chewable tablets, gummies, liquids, or powders. Dietary supplements may also be in other dosage forms, such as a bar, but if they are, information on the label of the dietary supplement may not represent the product as a conventional food or a sole item of a meal or diet.

For purposes of the present invention, the term “dopamine antagonist” or the term “dopamine receptor antagonist” refers to one of a group of compounds that block or inhibit the binding of dopamine to dopamine receptors. Dopamine is a neurotransmitter that is found in the brains of animals, including humans, and is essential for proper nerve signal transmission.

For purposes of the present invention, the term “dosage” refers to the administering of a specific amount, number, and frequency of doses over a specified period of time. Dosage implies duration. A “dosage regimen” is a treatment plan for administering a drug over a period of time.

For purposes of the present invention, the term “dosage form,” the term “form,” or the term “unit dose” refers to a method of preparing pharmaceutical products in which separate doses of medications are prepared and delivered. Dosage forms typically involve a mixture of active drug components and nondrug components (excipients), along with other non-reusable material that may not be considered either ingredient or packaging.

For purposes of the present invention, the term “dose” refers to a specified amount of medication taken at one time.

For purposes of the present invention, the term “effective amount” or “effective dose” or grammatical variations thereof refers to an amount of an agent sufficient to produce one or more desired effects. The effective amount may be determined by a person skilled in the art using the guidance provided herein.

For purposes of the present invention, the term “energy balance” refers to a situation when energy intake equals energy expenditure and when body energy (generally equivalent to body weight) is stable. The basic components of energy balance include energy intake, energy expenditure, and energy storage. Body weight can change only when energy intake is not equal to energy expenditure over a given period of time.

For purposes of the present invention, the term “food addiction” refers to an individual or subject being addicted to junk food in the same way as drug addicts are addicted to drugs.

For purposes of the present invention, the term “inverse agonist” refers to a compound that binds to the same receptor as an agonist but induces a pharmacological response opposite to that of the agonist. A prerequisite for an inverse agonist response is that the receptor must have a constitutive (also known as intrinsic or basal) level activity in the absence of any ligand. An agonist increases the activity of a receptor above its basal level, whereas an inverse agonist decreases the activity below the basal level. A neutral antagonist has no activity in the absence of an agonist or inverse agonist but can block the activity of either. The efficacy of a full agonist is by definition 100%, a neutral antagonist has 0% efficacy, and an inverse agonist has <0% (i.e., negative) efficacy.

For purposes of the present invention, the term “ligand” refers to a substance (usually a small molecule) that forms a complex with a biomolecule to serve a biological purpose. In protein-ligand binding, the ligand is usually a signal-triggering molecule, binding to a site on a target protein. Ligand binding to a receptor protein (receptor) alters the receptor's chemical conformation (three-dimensional shape). The conformational state of a receptor determines its functional state. Ligands include substrates, inhibitors, activators, and neurotransmitters.

For purposes of the present invention, the term “medical therapy” refers to prophylactic, diagnostic and therapeutic regimens carried out in vivo or ex vivo on humans or other mammals.

For purposes of the present invention, the term “mg/kg” refers to the dose of a substance administered to an individual or a subject in milligrams per kilogram of body weight of the individual or the subject.

For purposes of the present invention, the term “nutraceutical” refers to compounds and compositions that are useful in both the nutritional and pharmaceutical field of application. Thus, nutraceutical compositions of the present invention may be used as supplement to food and beverages, and as pharmaceutical formulations for enteral or parenteral application which may be solid formulations such as capsules or tablets, or liquid formulations, such as solutions or suspensions. In some embodiments of the present invention, nutraceutical compositions may also comprise food and beverages containing therapeutically effective amount of one or more non-selective opioid receptor antagonists, CNS stimulants, pharmaceutically acceptable analogs, salts or hydrates of the one or more respective non-selective opioid receptor antagonists, CNS stimulants, as well as supplement compositions, for example dietary supplements.

For purposes of the present invention, the term “obesity” and the term “obesity associated disease” refer to a medical condition in which excess body fact has accumulated to the extent that it may have a negative effect on health, leading to reduced life expectancy and/or increased health problems. In Western countries, people are considered obese when their body mass index (BMI) exceeds 30 kg/m2, with the range 25-30 kg/m2 defined as overweight. Obesity increases the likelihood of various diseases, particularly heart disease, type 2 diabetes, obstructive sleep apnea, certain types of cancer, and osteoarthritis. Obesity is most commonly caused by a combination of excessive food energy intake, lack of physical activity, and genetic susceptibility. In a few cases obesity are caused primarily by genes, endocrine disorders, medications, or psychiatric illness.

For purposes of the present invention, the term “parenteral route” refers to the administration of a composition, such as a drug in a manner other than through the digestive tract.

For purposes of the present invention, the term “pharmaceutically acceptable” refers to a compound or drug approved or approvable by a regulatory agency of a federal or a state government, listed or listable in the U.S. Pharmacopeia or in other generally recognized pharmacopeia for use in mammals, including humans.

For purposes of the present invention, the term “pharmaceutically acceptable salt” refers to those salts of compounds that are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well-known in the art. They may be prepared in situ when finally isolating and purifying the compounds of the invention, or separately by reacting them with pharmaceutically acceptable non-toxic bases or acids, including inorganic or organic bases and inorganic or organic acids. Pharmaceutically acceptable salts may be obtained using standard procedures well known in the art, for example by mixing a compound of the present invention with a suitable acid, for instance an inorganic acid or an organic acid. Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, and diethanolamine salts.

For purposes of the present invention, the term “pharmaceutical composition” refers to a product comprising one or more active ingredients, and one or more other components such as carriers, stabilizers, diluents, dispersing agents, suspending agents, thickening agents, and/or excipients, etc. A pharmaceutical composition includes enough of the active object compound to produce the desired effect upon the progress or condition of diseases and facilitates the administration of the active ingredients to an organism. Multiple techniques of administering the active ingredients exist in the art including, but not limited to: topical, ophthalmic, intraocular, periocular, intravenous, oral, aerosol, parenteral, and administration. By “pharmaceutically acceptable,” it is meant the carrier, diluent or excipient must be compatible with the other ingredients of the formulation and not deleterious to the recipient thereof, i.e., the subject.

For purposes of the present invention, the term “pharmaceutical formulation” and the term “drug formulation” refer to a mixtures or a structure in which different chemical substances, including the active drug, are combined to form a final medicinal product, such as a sterile product, a capsule, a tablet, a powder, a granule, a solution, an emulsion, a topical preparation, a non-conventional product such as semi-solid or sustained-release preparations, liquid, etc. Pharmaceutical formulation is prepared according to a specific procedure, a “formula.” The drug formed varies by the route of administration. For example, oral drugs are normally taken as tablet or capsules.

For purposes of the present invention, the term “polypill” refers to a drug product in pill form (i.e., tablet or capsule) that combines multiple active pharmaceutical ingredients. A polypill comprises multiplicity of distinct drugs in a given “pill.” It may be manufactured as a fixed-dose combination drug product.

For purposes of the present invention, the term “rewarding effect” refers to the effect of the induced neural activity that leads the rat or mouse to seek out and re-initiate the stimulation. A drug is rewarding if the rat or mouse displays a preference for the drug-associated compartment and aversive if the rat or mouse displays a preference for the alternative compartment.

For purposes of the present invention, the term “subject” or the term “individual” refers to an animal, for example, a mammal, such as a human, who has been the object of treatment, observation or experiment.

For purposes of the present invention, the term “synergistic effect” refers to a combined effect when two or more substances or biological structures interact resulting in an overall effect that is greater than the sum of separate effects of any of the two or more substances or biological structures. For example, a synergistic effect of two therapeutic compounds means that an effect of administering two therapeutic compounds in combination is greater than the sum of each effect when each of the two therapeutic compounds is administered alone.

For purposes of the present invention, the term “tablet” refers to a pharmaceutical dosage form. A tablet comprises a mixture of active substances and excipients, usually in powder form, pressed or compacted from a powder into a solid dose. The excipients can include diluents, binders or granulating agents, glidants and lubricants to ensure efficient tableting; disintegrants to promote tablet break-up in the digestive tract; sweeteners or flavors to enhance taste; and pigments to make the tablets visually attractive. A polymer coating is often applied to make the tablet smoother and easier to swallow, to control the release rate of the active ingredient, to make it more resistant to the environment (extending its shelf life), or to enhance the tablet's appearance. The disintegration time can be modified for a rapid effect or for sustained release. For example, Some tablets are designed with an osmotically active core, surrounded by an impermeable membrane with a pore in it. This allows the drug to percolate out from the tablet at a constant rate as the tablet moves through the digestive tract. Tablets can also be coated with sugar, varnish, or wax to disguise the taste. A tablet in an embodiment of the present may comprise a tablet without or with one or more coatings. A tablet may also have one or more layers. A tablet may be mini tablet, a meltable table, chewable tablet, an effervescent tablet or an orally disintegrating tablet.

For purposes of the present invention, the term “target” refers to a living organism or a biological molecule to which some other entity, like a ligand or a drug, is directed and/or binds. For example, “target protein” may a biological molecule, such as a protein or protein complex, a receptor, or a portion of a biological molecule, etc., capable of being bound and regulated by a biologically active composition such as a pharmacologically active drug compound.

For purposes of the present invention, the term “time release,” the term “extended-release,” or “controlled-release” refers to a preparation that prolongs absorption of drugs with short half-lives, thereby allowing longer dosing intervals while minimizing fluctuations in serum drug levels. For example, a drug in a time release pill tables or capsules drug may be dissolved over time and be released slower and steadier into the bloodstream while having the advantage of being taken at less frequent intervals than immediate-release formulations of the same drug.

For purposes of the present invention, the term “therapeutically effective amount” refers to the amount of a compound or composition that, when administered to a subject for treating a disease or disorder, or at least one of the clinical symptoms of a disease or disorder, is sufficient to affect such treatment of the disease, disorder, or symptom. A “therapeutically effective amount” may vary depending, for example, on the compound, the disease, disorder, and/or symptoms of the disease or disorder, severity of the disease, disorder, and/or symptoms of the disease or disorder, the age, weight, and/or health of the subject to be treated, and the judgment of the prescribing physician. An appropriate amount in any given instance may be readily ascertained by those skilled in the art or capable of determination by routine experimentation.

For purposes of the present invention, the term “transdermal patch” refers to a medicated adhesive patch that is placed on the skin to deliver a specific dose of medication through the skin and into the bloodstream. A transdermal patch may provide a controlled release of the medication into the body of a subject.

For purposes of the present invention, the term “treating” or the term “treatment” of any disease or disorder refers to arresting or ameliorating a naturally occurring condition (for example, as a result of aging), disease, disorder, or at least one of the clinical symptoms of a disease or disorder, reducing the risk of acquiring a disease, disorder, or at least one of the clinical symptoms of a disease or disorder, reducing the development of a disease, disorder or at least one of the clinical symptoms of the disease or disorder, or reducing the risk of developing a disease or disorder or at least one of the clinical symptoms of a disease or disorder. “Treating” or “treatment” also refers to slowing the progression of a condition, inhibiting the disease or disorder, either physically, (e.g., stabilization of a discernible symptom), physiologically, (e.g., stabilization of a physical parameter), or both, and to inhibiting or slowing the progression of at least one physical parameter which may or may not be discernible to the subject. In some embodiments of the present invention, the terms “treating” and “treatment” refer to delaying the onset of the progression of the disease or disorder or at least one or more symptoms thereof in a subject who may be exposed to or predisposed to a disease or disorder even though that subject does not yet experience or display symptoms of the disease or disorder. The term “treatment” as used herein also refers to any treatment of a subject, such as a human condition or disease, and includes: (1) inhibiting the disease or condition, i.e., arresting the development or progression of the disease or condition, (2) relieving the disease or condition, i.e., causing the condition to regress, (3) stopping the symptoms of the disease, and/or (4) enhancing the conditions desired.

For purposes of the present invention, the term “vehicle” refers to a substance of no therapeutic value that is used to convey an active medicine for administration.

For purposes of the present invention, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

For purposes of the present invention, a value or property is “based” on a particular value, property, the satisfaction of a condition or other factor if that value is derived by performing a mathematical calculation or logical operation using that value, property or other factor.

### DESCRIPTION

Embodiments of the present invention provide a treatment for obesity via the use of combinations of one or more opioid antagonists and one or more CNS stimulants.

Obesity in pediatric and adult populations remains a major public health and public policy concern all over the world. In Western countries, people are considered obese when their body mass index (BMI) exceeds 30 kg/m², with the range 25-30 kg/m² defined as overweight. The US Centers for Disease estimate that 37.5% of adults and about 17% of children and adolescents (ages 2-19) are obese in the USA. Obesity is linked to elevated serum cholesterol, type 2 diabetes and high blood pressure, all of which raise the risk of heart attacks and stroke. In addition, obesity is an independent risk factor in cardiovascular disease and cancer. Thus, the consequences of obesity are serious, affecting multiple organ systems and contributing to reduced lifespan and poor quality of life.

Obesity is often considered to be caused by a combination of excessive food energy intake, lack of physical activity, and genetic susceptibility. Obesity may also be viewed in terms of energy balance. Energy intake and expenditure are the two sides of the energy balance coin. Dopamine is a critical neurotransmitter in the brain circuits that control energy balance and plays a key role in the brain reward circuit.

CNS stimulant compounds such as amphetamines and methylphenidate (MPH) target the dopaminergic system in the brain and suppress food intake.^{1,2} Therefore, CNS stimulants, or stimulants, are highly effective anti-obesity drugs. As an example, amphetamines ware approved and successfully used as weight loss drugs in early to mid-20th century.^{1,3} However, concerns about the abuse potential of stimulant compounds led to the abandonment of stimulants in subsequent years. Although a number of nonaddictive drugs currently dominate the anti-obesity drug landscape, none is superior to stimulants in terms of the ability to suppress food intake and produce weight loss.^{1,2,3}

An overwhelming body of research shows conclusively that stimulants administered at therapeutic doses are safe and effective in pediatric and adult populations in the management of Attention-Deficit Hyperactivity Disorder (ADHD).^{4,5} However, stimulants are prone to abuse.^{6,7,8,9,10} Although stimulant medications are safe and have a proven record of effectiveness against obesity, the short- or long-term use as anti-obesity drugs is essentially prohibited. In addition, some stimulants produce dysphoria, a feeling of discomfort or uneasiness. This side effect of stimulants is also a barrier for continued use of stimulants. If the side effects of stimulants such as potency of abuse and dysphoria can be eliminated or significantly mitigated, this powerful class of anti-obesity drugs can be brought back safely to clinical practice.

A number of studies suggest that rapid elevation of MPH levels in the blood and brain that occurs following intranasal or oral administration of supra-therapeutic doses is a key requirement for development of MPH-associated euphoria, reinforcement, and addiction. The principal molecular targets of MPH in the central nervous system (CNS) are dopamine and noradrenaline, however, at sufficiently high-doses MPH also activates the μ opioid receptor (MOPR) in the striatum and nucleus accumbens, brain regions associated with reward circuity.¹¹ Opioid receptors are a group of G protein-coupled receptors with opioids as ligands. In the brain opioid receptors fall into 3 types: Mu (μ), delta (δ) and kappa (κ). Caudate-putamen, nucleus accumbens, frontal cortex and ventral midbrain, all of which are intricately involved in the reward and addiction circuitry, are enriched in these receptors. Each receptor is believed to facilitate different aspects of reward circuits via interactions with opioids, endorphins and neurotransmitters including dopamine Mu-opioid-receptors (MOPR) are a key molecular switch triggering brain reward systems and potentially initiating addictive behaviors.

MPH is not known to directly activate MOPR. Dopamine D1-receptor activation is found to be an essential step in the activation of MOPR by MPH.¹¹ Since the rewarding effects of high doses of MPH are associated with MOPR activation, blocking the MOPR may mitigate the reinforcing effects of MPH.¹¹

Accordingly, embodiments of the present invention provide a method for treating obesity for a subject having a need thereof by the administration of a combination of one or more non-selective opioid receptor antagonists and one or more CNS stimulants that have an established anti-obesity track record. The one or more CNS stimulants includes one or more stimulant-like compounds that act like stimulants and block the re-uptake of multiple neurotransmitters, such as dopamine, serotonin and noradrenaline, etc. The one or more none-selective opioid receptor antagonists may block the MOPR and prevent MOPR activation and thereby block rewarding effects induced by stimulants such as MPH. As a result, the combination of one or more non-selective opioid receptor antagonists and one or more stimulants with an established anti-obesity track record will render the combination highly efficacious and yet abuse free for the treatment of obesity. It is found that an opioid receptor antagonist administered alone can produce weight loss by acting on the food reward system of the brain.^{12,13,14} Therefore, the combination of the stimulants and opioid receptor antagonist will produce synergistic effects and will be more potent than the effect of either compound used alone.

A subject who may receive the treatments in embodiments of the present invention may be obese or overweight, may have food addiction, may have a binge-eating disorder, or may engage in a binge eating behavior. A subject may also be an individual who is at risk of developing a food addiction or developing a binge eating behavior.

According to embodiments disclosed herein, a combination is administered to a subject having a need thereof to treat obesity, and the combination comprises two or more compounds, wherein the two or more compounds comprise: a therapeutically effective amount of one or more non-selective opioid receptor antagonists and/or pharmaceutically acceptable analogs, salts or hydrates of the one or more opioid receptor antagonists; and a therapeutically effective amount of one or more CNS stimulants and/or pharmaceutically acceptable analogs, salts, or hydrates of the one or more CNS stimulants. In some embodiments, the one or more CNS stimulants may include, but are not limited to, methylphenidate, amphetamine, analeptic. The one or more CNS stimulants in some embodiments may also comprise one or more stimulant-like compounds that act like stimulants and block the re-uptake of multiple neurotransmitters, dopamine, serotonin and noradrenaline, etc. In some embodiments of the present invention, the one or more non-selective opioid receptor antagonists may comprise naltrexone.

Dysphoria induced by some stimulants is associated with the activation of kappa (K) opioid receptors by the stimulants. Through antagonizing the kappa opioid receptors, an opioid receptor antagonist such as naltrexone mitigates or eliminates the side effect of dysphoria caused by a stimulant. Therefore, the combination of stimulants with opioid receptor antagonists may prevent or reduce the dysphoria produced when stimulants are administered alone. Prevention or reduction of dysphoria may promote continued intake of the stimulant plus opioid receptor antagonist combination.

According to embodiments of the present invention, the two or more compounds comprised in a combination may be administered to a subject simultaneously, concurrently, or sequentially. For example, a therapeutically effective amount of one or more non-selective opioid receptor antagonists and/or pharmaceutically acceptable analogs, salts or hydrates of the one or more opioid receptor antagonists and a therapeutically effective amount of one or more CNS stimulants and/or pharmaceutically acceptable analogs, salts, or hydrates of the one or more CNS stimulants may be administered to a subject at the same time. A therapeutically effective amount of one or more non-selective opioid receptor antagonists and/or pharmaceutically acceptable analogs, salts or hydrates of the one or more opioid receptor antagonists may also be administered to a subject having a need thereof prior to an administration of a therapeutically effective amount of one or more CNS stimulants and/or pharmaceutically acceptable analogs, salts, or hydrates of the one or more CNS stimulants. In one embodiment, a therapeutically effective amount of one or more non-selective opioid receptor antagonists and/or pharmaceutically acceptable analogs, salts or hydrates of the one or more opioid receptor antagonists is administered to a subject having a need thereof 30 minutes prior to an administration of a therapeutically effective amount of one or more CNS stimulants and/or pharmaceutically acceptable analogs, salts, or hydrates of the one or more CNS stimulants.

In some embodiments, combinations disclosed herein may be administered to a subject have a need thereof with a pharmaceutical carrier or a nutraceutical carrier. In some embodiments, combinations disclosed herein may be orally administered to a subject. In some embodiments, combinations disclosed herein may be administered to a subject via a parenteral route such as intranasal route, transdermal delivery route, etc.

Embodiments disclosed herein provide a treatment of obesity by the administration of a combination comprising two or more compounds to a subject having a need thereof, wherein the two or more compounds comprise: a therapeutically effective amount of naltrexone and/or pharmaceutically acceptable analogs, salts or hydrates of naltrexone; and a therapeutically effective amount of MPH and/or pharmaceutically acceptable analogs, salts, or hydrates of MPH. Naltrexone, in combination with MPH, may abolish the abuse potential of MPH and eliminate or mitigate MPH's dysphoric effect. In one embodiment, the combination of naltrexone and MPH in a therapeutically effective amount is administered to a subject who has, or is at risk of developing, a food addiction. In another embodiment, the combination of naltrexone and MPH in a therapeutically effective amount is administered to a subject who has, or is at risk of developing, a binge-eating disorder, or engages in a binge eating behavior.

In some embodiments, a combination of naltrexone and MPH in therapeutically effective amount may be administered to a subject having a need thereof with a pharmaceutical carrier or a nutraceutical carrier. In some embodiments, a combination of naltrexone and MPH in therapeutically effective amount may be orally administered to a subject. In some embodiments, a combination of naltrexone and MPH in therapeutically effective amount may be administered to a subject via a parenteral route, such as intranasal route, transdermal delivery route, etc.

According to embodiments of the present invention, naltrexone and MPH, and/or pharmaceutically acceptable analogs, salts, or hydrates of naltrexone and MPH, in a combination may be administered to a subject simultaneously, concurrently, or sequentially. For example, a therapeutically effective amount of naltrexone and MPH may be administered to a subject at the same time. A therapeutically effective amount of naltrexone and/or pharmaceutically acceptable analogs, salts, or hydrates of naltrexone may also be administered to a subject having a need thereof prior to an administration of a therapeutically effective amount of MPH and/or pharmaceutically acceptable analogs, salts, or hydrates of MPH. In one embodiment, a therapeutically effective amount of naltrexone and/or pharmaceutically acceptable analogs, salts or hydrates of naltrexone is administered to a subject having a need thereof 30 minutes prior to the administration of a therapeutically effective amount of MPH and/or pharmaceutically acceptable analogs, salts, or hydrates of MPH.

The dose of each therapeutic compound in the combination disclosed herein that is administered to a subject may be adjusted to provide an optimal therapeutic response. The specific dose level for any particular subject may vary depending upon a variety of factors such as age, body weight, general health, sex, diet, the time of administration the rate of excretion, the severity of a particular disease or disorder being treated, the form of administration, etc. The therapeutically effective amount of each of the two or more compounds in the combination may be flexible in a wide variety with regard to a specific compound that targets opioid receptors and to a specific compound that stimulates rewarding effect. The dose of each of the two or more compounds in the combination may be determined by one skilled in the art and the dosage regime may be determined according to the situation of a subject having a need thereof.

In some embodiments, naltrexone may be administered to a human subject at a daily dose from about 0.5 mg/kg to about 1.5 mg/kg, and MPH may be administered to a human subject at a daily dose from about 0.75 mg/kg to about 2 mg/kg. The amount of naltrexone and MPH in a combination may be flexible according to need. For example, in one embodiment, a daily dose of combination may comprise about 0.5 mg/kg of naltrexone and about 0.75 mg/kg of MPH. In another embodiment, a daily dose of combination may comprise about 1.5 mg/kg naltrexone and about 1 mg/kg of MPH. The examples are not exclusive. The therapeutically effective amount of MPH and naltrexone in a combination administered to a human subject per day may be any combination of about 0.75 to about 2 mg/kg of MPH and about 0.5 to 1.5 mg/kg of naltrexone, depending on the need for providing optimal therapeutic effect. In some situation, the daily amount of MPH in a combination may be less than about 0.75 mg/kg or more than 2 mg/kg, and the daily amount of naltrexone may be less than 0.5 mg/kg or more than 1.5 mg/kg, depending on the need for providing optimal therapeutic responses.

According to embodiments of the present invention, a product comprising at least one dosage of a combination for treating obesity, wherein the combination comprises a therapeutically effective amount of two or more compounds, and wherein the two or more compounds comprise: one or more non-selective opioid receptor antagonists and/or pharmaceutically acceptable analogs, salts or hydrates of the one or more opioid receptor antagonists; and one or more stimulants and/or pharmaceutically acceptable analogs, salts, or hydrates of the one or more stimulants. In one embodiment, the one or more stimulants may comprise methylphenidate. In another embodiment, the one or more stimulants in the product comprise amphetamine. The one or more CNS stimulants in the product in some embodiments may also comprise one or more stimulant-like compounds that act like stimulants and block the re-uptake of multiple neurotransmitters, such as dopamine, serotonin and noradrenaline, etc. In one embodiment, the one or more CNS stimulants in the product comprise analeptic. In one embodiment, the one or more non-selective opioid antagonists in the product comprise naltrexone.

In some embodiments, the product comprises an orally administrable nutraceutical composition and wherein the nutraceutical composition comprises one or more non-selective opioid receptor antagonists and/or pharmaceutically acceptable analogs, salts or hydrates of the one or more opioid receptor antagonists; and one or more CNS stimulants and/or pharmaceutically acceptable analogs, salts, or hydrates of the one or more CNS stimulants in pharmaceutically effective amount. In some embodiments, combinations in the embodiments may be also formulated in a pharmaceutical composition. According to embodiments, compounds in the combination disclosed herein may be formulated as a fixed-dose combination, or as a non-fixed combination. The combination may be in the dosage form of a tablet, a softgel, a capsule, a caplet, a polypill, a chewable tablet, a gummy, or a hard capsule, a transdermal patch, etc.

In some embodiment, the combination may be co-packaged drug product in which the therapeutically effective amount of one or more non-selective opioid receptor antagonists and/or pharmaceutically acceptable analogs, salts or hydrates of the one or more opioid receptor antagonists and the therapeutically effective amount of one or more CNS stimulants and/or pharmaceutically acceptable analogs, salts, or hydrates of the one or more CNS stimulants are in separate dosage forms packaged together in a single package or as a unit. In some embodiments, product may be a fixed dose combination in which the therapeutically effective amount of one or more non-selective opioid receptor antagonists and/or pharmaceutically acceptable analogs, salts or hydrates of the one or more non-selective opioid receptor antagonists and the therapeutically effective amount of one or more CNS stimulants and/or pharmaceutically acceptable analogs, salts, or hydrates of the one or more CNS stimulants are combined in a single dosage form, such as a tablet, a softgel, a capsule, a caplet, a syrup, a chewable tablet, a gummy, or a hard capsule, etc.

Embodiments disclosed herein also provide a product comprising at least one dosage of a combination for treating obesity. The combination in the product comprises a therapeutically effective amount of naltrexone and/or pharmaceutically acceptable analogs, salts, or hydrates of naltrexone, and a therapeutically effective amount of MPH and/or pharmaceutically acceptable analogs, salts, or hydrates of MPH. In some embodiments, the combination may be co-packaged drug product in which the therapeutically effective amount of naltrexone and/or pharmaceutically acceptable analogs, salts, or hydrates of naltrexone and the therapeutically effective amount of MPH and/or pharmaceutically acceptable analogs, salts, or hydrates of MPH are in separate dosage forms packaged together in a single package or as a unit.

In some embodiments, the product comprises an orally administrable nutraceutical composition and wherein the nutraceutical composition comprises the two or more compounds. The two or more compounds in this product comprise a therapeutically effective amount of naltrexone and/or pharmaceutically acceptable analogs, salts, or hydrates of naltrexone and a therapeutically effective amount of MPH and/or pharmaceutically acceptable analogs, salts, or hydrates of MPH. In some embodiments, combinations in the embodiments may be also formulated in a pharmaceutical composition.

According to embodiments, compounds in the combination disclosed herein may be formulated as a fixed-dose combination, or as a non-fixed combination. The combination may be in the dosage form of a tablet, a softgel, a capsule, a caplet, a polypill, a syrup, a chewable tablet, a gummy, or a hard capsule, a transdermal patch, etc.

According to an embodiment, a dosage of a combination for treating obesity may be formulated as a capsule that comprises an insoluble core encased within a soluble shell, wherein the insoluble core comprises naltrexone and/or pharmaceutically acceptable analogs, salts, or hydrates of naltrexone, wherein the soluble shell comprises MPH and/or pharmaceutically acceptable analogs, salts, or hydrates of MPH, and wherein when the capsule is taken orally, only MPH and/or pharmaceutically acceptable analogs, salts, or hydrates of MPH are absorbed and the insoluble core passes through the gastro-intestinal tract intact. When the capsule is used therapeutically, the naltrexone is not absorbed into the system, therefore such application may alleviate concerns about potential side effects of high doses of naltrexone, including actions at kappa and delta opioid receptors, as well as naltrexone's potential interaction with therapeutic actions of MPH. If the capsule is crushed for abuse, the naltrexone is released and would mitigate the abuse potential of MPH.

Similarly, according to embodiments, such insoluble core encased within a soluble shell may comprise one or more non-selective opioid receptor antagonists and/or pharmaceutically acceptable analogs, salts or hydrates of the one or more non-selective opioid receptors, and the soluble shell may comprise one or more CNS stimulants and/or pharmaceutically acceptable analogs, salts, or hydrates of the one or more CNS stimulants. When a capsule comprising the insoluble core encased within the soluble shell is taken orally, only the one or more CNS stimulants and/or pharmaceutically acceptable analogs, salts, or hydrates of the one or more CNS stimulants are absorbed and the insoluble core passes through the gastro-intestinal tract intact.

According to embodiments, product of the combination may be controlled release. In some embodiments, compounds in a combination may be formulated in different dosage form and/or be released at different time. For example, in some embodiments: the one or more non-selective opioid receptor antagonists and/or pharmaceutically acceptable analogs, salts or hydrates of the one or more non-selective opioid receptors are in an immediate release dosage form; and the one or more CNS stimulants and/or pharmaceutically acceptable analogs, salts, or hydrates of the one or more CNS stimulants are in a delayed-release dosage form. In one embodiment, the naltrexone and/or pharmaceutically acceptable analogs, salts, or hydrates of naltrexone are formulated in an immediate release dosage form, and methylphenidate and/or pharmaceutically acceptable analogs, salts, or hydrates of methylphenidate are formulated in a delayed-release dosage form. Compounds such as non-selective opioid receptors and CNS stimulants in separate dosage forms may be packaged together in a single package.

In some embodiment, the coating materials for the one or more CNS stimulants and/or pharmaceutically acceptable analogs, salts, or hydrates of the one or more CNS stimulants may be different from the coating materials for the one or more non-selective opioid receptor antagonists and/or pharmaceutically acceptable analogs, salts or hydrates of the one or more non-selective opioid receptors, so that the one or more CNS stimulants and/or pharmaceutically acceptable analogs, salts, or hydrates of the one or more CNS stimulants may be released later than the one or more non-selective opioid receptor antagonists and/or pharmaceutically acceptable analogs, salts or hydrates of the one or more non-selective opioid receptors. For example, in a combination, different coating materials for MPH and naltrexone are applied, and as a result, upon administration, MPH is released later than naltrexone. In one embodiment, MPH may be released about 30 minutes later than naltrexone.

According to embodiments, compounds in a combination may be formulated in separate dosage forms and can be mixed together before an administration of the combination to a subject having a need thereof. In one embodiment, the therapeutically effective amount of naltrexone and/or pharmaceutically acceptable analogs, salts, or hydrates of naltrexone are formulated in a dosage form separated from the dosage form of the therapeutically effective amount of methylphenidate and/or pharmaceutically acceptable analogs, salts, or hydrates of methylphenidate, and wherein compositions in each dosage form can be mixed together before an administration of the combination to a subject having a need thereof.

Embodiments disclosed herein also provide some treatment delivery apparatus that comprise treatment carrier devices and at least one dosage of a combination disclosed herein contained in the each of the treatment devices for treating obesity. The combination may comprise a therapeutically effective amount of one or more non-selective opioid receptor antagonists and/or pharmaceutically acceptable analogs, salts or hydrates of the one or more non-selective opioid receptor antagonists; and a therapeutically effective amount of one or more CNS stimulants and/or pharmaceutically acceptable analogs, salts, or hydrates of the one or more stimulants. In some embodiment, the one or more non-selective opioid receptor antagonists comprised in the combination contained in the treatment carrier device is naltrexone, and the one or more CNS stimulants in the combination is methylphenidate.

According to embodiments, a treatment delivery apparatus for treating obesity may be a transdermal patch. The transdermal patch may comprise an adhesive patch and a combination disclosed herein. A combination disclosed herein may be deposited as one or more active layer and be embedded into the adhesive patch. The combination disclosed herein may be one or more non-selective opioid receptor antagonists and/or pharmaceutically acceptable analogs, salts or hydrates of the one or more non-selective opioid receptor antagonists; and one or more CNS stimulants and/or pharmaceutically acceptable analogs, salts, or hydrates of the one or more CNS stimulants. The combination disclosed herein may also be a therapeutically effective amount of naltrexone and/or pharmaceutically acceptable analogs, salts, or hydrates of naltrexone; and a therapeutically effective amount of MPH and/or pharmaceutically acceptable analogs, salts, or hydrates of MPH. Such transdermal patch comprising the combination disclosed herein may be placed on a skin of a subject having a need thereof. The two or more compounds in the combination embedded in the adhesive patch may be released into the body of the subject. Such release may also be a controlled release. The two or more compounds such as naltrexone and MPH may be released at different rate.

The present invention is further defined in the following Examples. It should be understood that these Examples are given by way of illustration only. From the above discussion and these Examples, one skilled in the art can ascertain the essential characteristics of embodiments of the present invention. Without departing from the spirit and scope thereof, one skilled in the art can make various changes and modifications of the invention to adapt it to various usages and conditions. All publications, including patents and non-patent literature, referred to in this specification are expressly incorporated by reference herein.

### EXAMPLES

### Example 1

### Prior Exposure to Opioid Receptor Antagonist Attenuates High-Doses MPH-Induced CPP

This example illustrates that high doses of MPH induce conditioned place preference (CPP) and enhance μ opioid receptor (MOPR) activity in mouse model. Prior exposure to naltrexone attenuates high-dose MPH-induced CPP and decreases MOPR activity.

### Materials and Methods

C57/B6 mice are purchased from Charles River Laboratories. Only male mice are used.

### Conditioned Place Preference (CPP)

A three-chamber place preference apparatus is used. The apparatus has two equally sized (16.8×12 cm) preference chambers connected by a central chamber (7.2×12 cm), and is outfitted with sliding guillotine-style doors between each chamber. Photobeams connected to a computer system can record animal location and time spent in that location. The central chamber has a gray colored smooth floor. The preference chamber is either white with a mesh floor or black with a bar floor. The CPP procedure included three phases: Pre-conditioning, conditioning and test phases.

The pre-conditioning phase is performed on day 1 (two sessions daily, AM and PM). In each pre-conditioning session, mice are initially placed in the central gray chamber for 2 min and then allowed free access to the white and black chambers for 20 min. The time spent in each chamber is recorded.

For the next phase in the assay, the conditioning phase, the non-preferred chamber (i.e. the chamber in which less time is spent) is designated as the drug-paired chamber and the preferred chamber (i.e. the chamber in which more time was spent) is designated as the vehicle-paired chamber.

The conditioning phase is carried out on each of days 2-6. There are two conditioning sessions daily, morning session between 8 and 10 AM and afternoon session between 2 and 4 PM. There is one session each for vehicle-paired (saline as vehicle) and drug-paired (MPH as drugs) conditions on each day of the conditioning phase. The mice are administered saline or drug (i.p.) in the saline- or drug-paired sessions, respectively and placed in the central gray chamber for 2 min (to isolate injection effect to the central chamber) and then confined to the vehicle-or-drug-paired chamber, respectively for 30 min. On the first day of the conditioning phase, the mouse receive saline during the morning session and the drug in the afternoon session. The next day, the order of treatments is reversed: drug in the morning session and saline in the afternoon session. These two paradigms are alternated for the remainder of the conditioning phase. This conditioning strategy is used for all the experiments except those using naltrexone.

When naltrexone is used alone or co-administered with MPH, only one conditioning session per day (morning session between 8 and 10 AM) is performed. Saline is administered on the first day and naltrexone (or naltrexone+MPH) is administered the following day. Administrations of saline and naltrexone are alternated for each of the next 9 days (total duration of conditioning is 10 days). In the experiments using a drug co-administration strategy, naltrexone is administered 30 min prior to MPH.

During the test phase, (one session on the day after the conditioning phase) the mice are placed in the central gray chamber for 2 min and then given free access to the drug- and saline-paired chambers for 20 min. The time spent in each chamber is recorded. The difference between time spent in the drug-paired chamber during the test phase and pre-conditioning phases was calculated as the CPP score. Saline and the drugs are administered intraperitoneally. All the behavioral analyses are performed during the light phase of the light-dark cycle.

### [³⁵S]GTPγS Binding

Activity of the μ opioid receptor (MOPR) is assayed by using [35S]GTPγS binding on membrane preparations. The same mice that are used in the conditioned place preference assay are used for these assays Immediately upon completion of the behavioral assay, the mice are sacrificed by cervical dislocation and the brain is dissected rapidly. The brain is frozen in liquid nitrogen and 1.0 mm slices of the brain were prepared in the coronal plane using a tissue-slicing matrix (Model 15003; Ted Pella, Redding, Calif.). Caudate-putamen and nucleus accumbens are identified in the frozen slices based on anatomical landmarks and samples from these two brain regions are collected using a tissue punch. The samples are homogenized using teflon pestle in Eppendorf tubes (10 strokes) in 300 μl of homogenization buffer containing 25 mMTris/pH 7.4, 5 mMEDTA and 0.1 mMPMSF and kept on ice. Homogenate is diluted to 2.5 ml using the homogenization buffer and centrifuged at −350,000 g for 30 min. After washing 3 times with 50 mM Tris-HCl/pH 7.4, the pellets are re-suspended in 50 mM Tris-HCl/pH 7.4 containing 0.32 M sucrose, passed through a 26.5 G needle 3 times, frozen in dry ice/ethanol and stored in −80° C. until use. DAMGO is used to stimulate MOPR. The membranes (10 μg protein) are incubated in buffer (50 mM HEPES/pH 7.4, 100 mM NaCl, 5 mM MgCl2 and 1 mM EDTA/pH 8.0) containing [35S]GTPγS (100,000 dpm, 80 pM) and 100 μM GDP with or without DAMGO in a total volume of 0.5 ml for 60 min at 30° C.

Nonspecific binding is defined by incubation in the presence of 10 μM GTPγS. Nonspecific binding is found to be similar in the presence or absence of agonist and was subtracted from total stimulated and total basal binding. Bound and free [³⁵S]GTPγS are separated by filtration with GF/B filters under reduced pressure. Radioactivity on filters is determined by liquid scintillation counting. Nonspecific binding is subtracted from total stimulated and basal binding. The basal binding in the caudate-putamen ranges between 45.9±1.9 and 47.5±3.5 fmol/mg protein, while that in the nucleus accumbens is between 88.7±7.9 and 92.5±9.3 fmol/mg protein. MOPR activity is reported as % of baseline (unstimulated) activity:

$$\frac{\begin{matrix}DPM with agonist stimulated binding - \\ DPM of nonspecific binding\end{matrix}}{DPM basal ( without agonist ) - DPM nonspecific )} \times 100$$

### Data Analysis

Differences between two experimental groups are analyzed for statistical significance by using Student's t-test. Treatment effects on multiple groups are tested for significance by using one-way ANOVA and the differences among the groups are tested for significance by using Dunnett's multiple comparisons test.

### Results

### High Doses of MPH Induce Conditioned Place Preference

Reinforcing properties of MPH are influenced by the dose and route of administration, with parenteral high doses but not oral low doses leading to addiction. 0.75 mg/kg MPH administered to adult mice produces serum and brain concentrations of D-methylphenidate (the pharmacologically active isomer) that are equivalent to the levels seen in human subjects given oral therapeutic doses of MPH. Therefore, 0.75 mg/kg MPH is considered to be a low and safe dose with potentially therapeutic effect in human subjects.

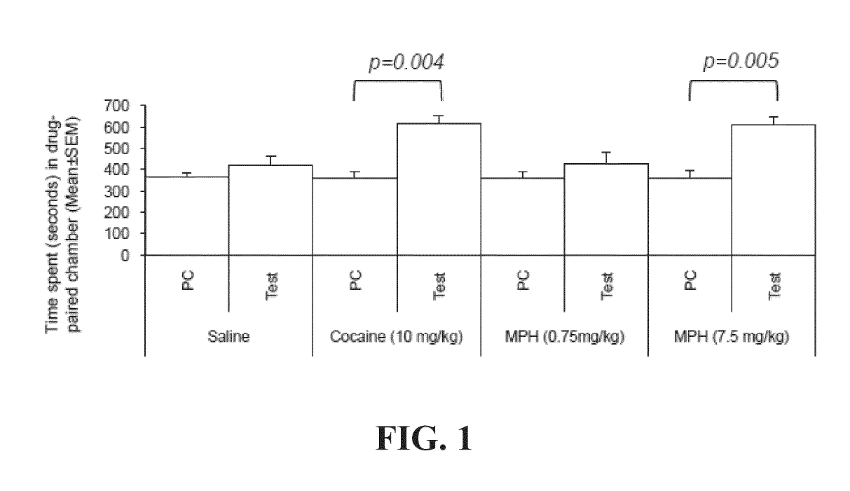

CPP paradigm is used to establish whether the high dose of MPH (7.5 mg/kg, 10 times the safe dose) could produce rewarding effects in a mouse model. Cocaine (10 mg/kg) is used as a positive control drug. Saline is used as a negative control. As shown in FIG. 1, mice exposed to cocaine or high-dose MPH (7.5 mg/kg) spend significantly longer period of time in the drug-paired chamber during the Test sessions compared to the pre-conditioning sessions, whereas the mice exposed to saline or low dose MPH (0.75 mg/kg) do not show significant differences in this measure. Mice exposed to the high-dose MPH show significant place preference (FIG. 1, t-test; p=0.005; n=6/group, n is the number of mice in the group), as do the mice exposed to cocaine (FIG. 1; t-test; p=0.004, n=6/group), while the mice exposed to either the low dose MPH or saline do not (FIG. 1; t-test, p>0.05).

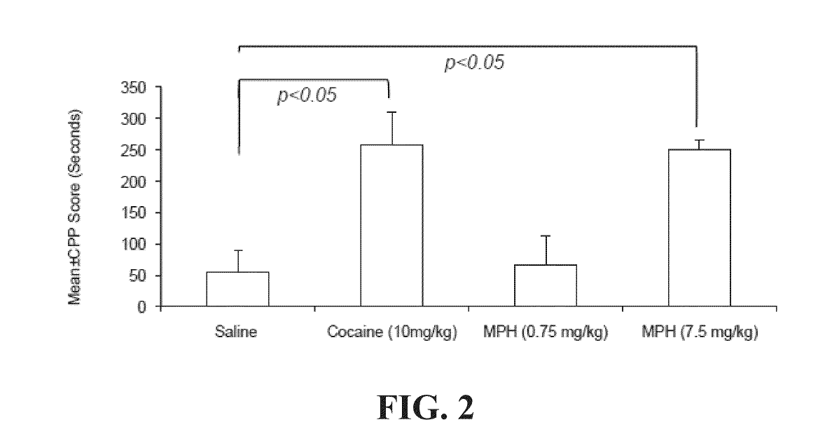

Multiple comparisons test shows significant (p<0.05) effects of cocaine and high-dose MPH treatment on the CPP score compared to the score in the saline-treated group and low dose MPH (0.75 mg/kg) groups. (FIG. 2). There is no significant difference between the saline and low dose MPH groups or between the cocaine and high-dose MPH groups. Thus, under these experimental conditions, high-dose MPH is essentially as rewarding as cocaine whereas the low dose and saline are equally ineffective in rewarding.

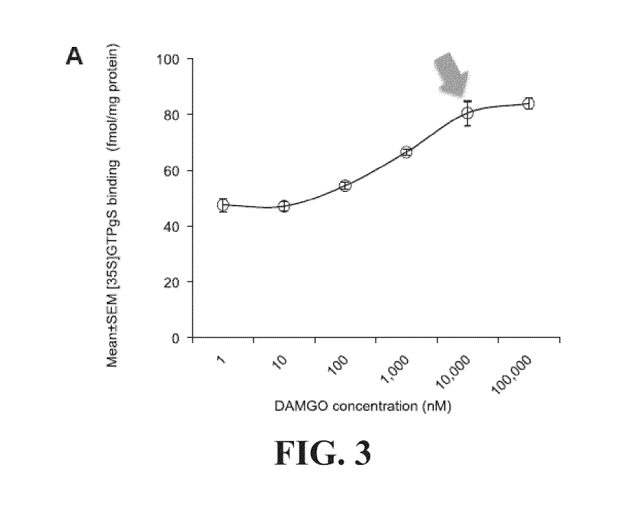

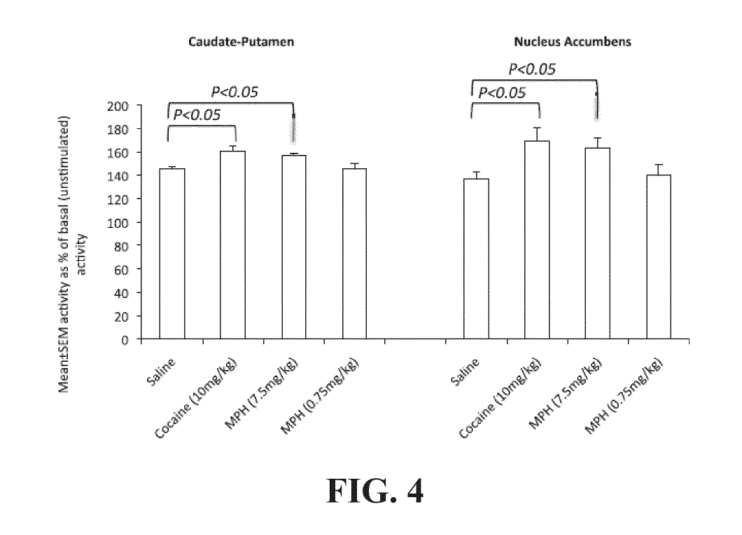

### High Doses of MPH Enhance μ Opioid Receptor Activity

[³⁵S]GTPγS binding in membrane preparations from the caudate-putamen is increased by a selective MOPR agonist DAMGO in a concentration-dependent manner with an EC₅₀ of about 1 and about 0.1 mM (FIG. 3). The maximal binding, which represented 1.75-fold of the basal level, is reached at 10 mM concentration (FIG. 3, arrow). 10 mM is the concentration of DAMGO used in the bindings assays shown in FIG. 4.

FIG. 4 shows a significant effect of the drug treatment on a selective MOPR agonist DAMGO-stimulated [³⁵S]GTPγS binding in the caudate-putamen (F=5.89; p=0.005; n=6 per group) and the nucleus accumbens (F=4.345, p=0.016; n=6 per group). The basal [³⁵S]GTPγS binding (i.e. unstimulated binding) is not significantly different among the different groups. Cocaine and high-dose MPH (7.5 mg/kg) groups show significant increases in MOPR activity compared to the saline group (p<0.05) in both the brain regions. There is no significant difference between the saline and low dose MPH (0.75 mg/kg) groups or between the cocaine and high-dose MPH groups in either brain region.

### Prior Exposure to Naltrexone Attenuates High-Dose MPH-Induced CPP

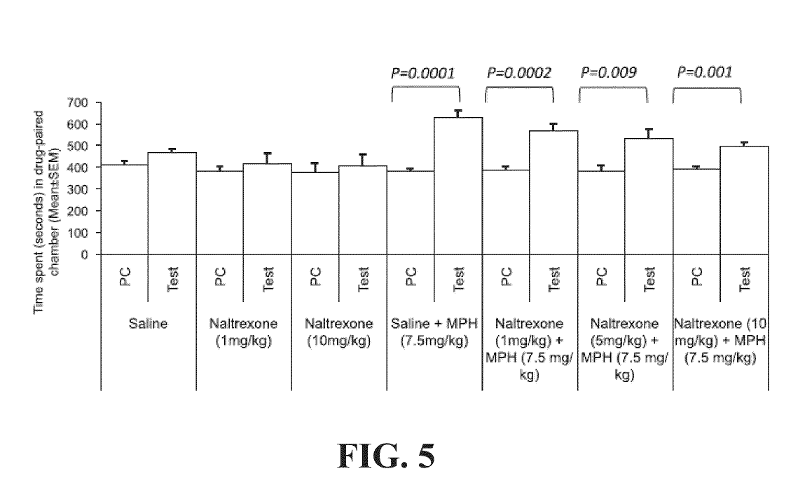

CPP assays are performed in which naltrexone is administered 30 min prior to MPH (7.5 mg/kg) at 1, 5, or 10 mg/kg. Saline, MPH (7.5 mg/kg) alone, and 1 and 10 mg/kg naltrexone alone are used as control. As shown in FIG. 5, mice exposed to high-dose MPH plus saline spend significantly longer period of time in the drug-paired chamber during the Test sessions compared to the pre-conditioning (PC) sessions. Neither saline alone nor naltrexone (1 or 10 mg/kg) alone produces significant changes in this measurement (FIG. 5. Naltrexone alone: t-tests, in each case, p>0.05 and n=6). When naltrexone (1, 5 or 10 mg/kg) is administered prior to MPH, in each case there is a significant difference between PC and Test sessions indicating that each drug treatment induces CPP. (FIG. 5, t-tests; 1 mg naltrexone+MPH, p=0.0002, n=11; 5 mg naltrexone+MPH, p=0.009, n=7; 10 mg naltrexone+MPH, p=0.001, n=7).

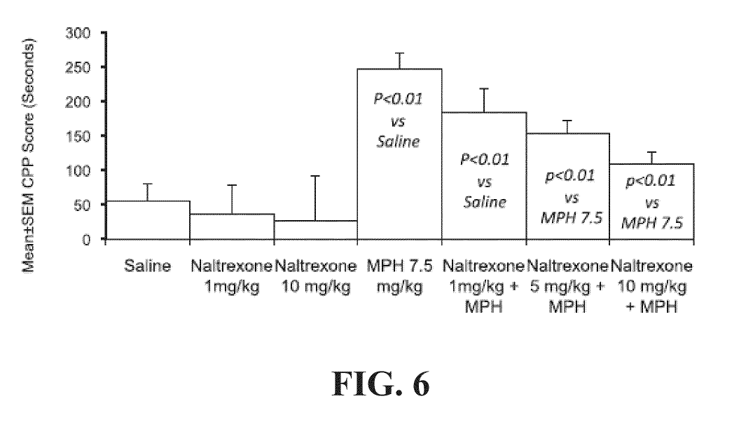

Multiple comparisons analysis of the CPP scores show that prior treatments with 1, 5 or 10 mg/kg naltrexone significantly decrease the CPP score compared to prior treatment with saline (FIG. 6). The decrease in the CPP score is naltrexone dose-dependent. In fact, the CPP score for the group that receives 5 mg/kg or 10 mg/kg naltrexone prior to MPH is not significantly different from that for the saline group (FIG. 6).

Multiple comparison test shows that the CPP score produced by the high-dose MPH (7.5 mg/kg) is significantly greater than that produced by the saline control (p<0.01). However, the CPP scores of neither the 5 mg naltrexone+MPH nor the 10 mg naltrexone+MPH groups are significantly different (FIG. 6; p>0.05) from the saline control groups. This result suggests that these two doses of naltrexone inhibit high-dose MPH-induced place preference. The CPP scores for the 1 mg naltrexone+MPH group are significantly higher than the saline control group (FIG. 6; p<0.01). These data show that blocking opioid receptors using naltrexone prior to MPH administration can significantly attenuate rewarding effects of MPH.

### Prior Exposure to Naltrexone Attenuates High-Dose MPH-Induced Upregulation of MOPR Activity

MOPR activity following the CPP assay in which MPH+naltrexone combinations are employed is analyzed. Multiple comparisons analysis shows that the MOPR activity in the MPH-treated group is significantly higher than that in the saline group. MOPR activity in the MPH+naltrexone (10 mg/kg) group is significantly lower than that in the saline or MPH groups.

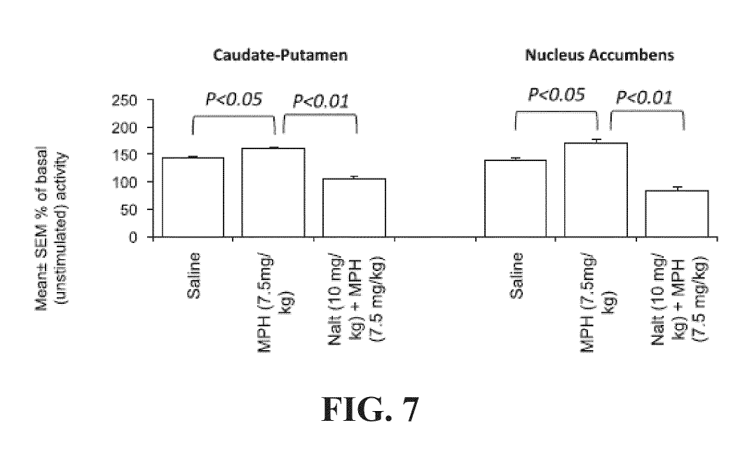

At the end of the CPP assay, the caudate-putamen and nucleus accumbens from each group of mice are collected and MOPR activity is assayed by using [³⁵S]GTPγS binding. The basal [³⁵S]GTPγS binding is not significantly different among the different groups. Comparison between the saline controls and high-dose MPH (7.5 mg/kg) groups show that the MOPR activity in the MPH-treated group is significantly higher than that in the saline group. (FIG. 7). MOPR activity in the MPH+naltrexone (10 mg/kg) group is significantly lower than that in the saline or MPH groups. (caudate-putamen: F=66.17, p<0.001; n=8; and the nucleus accumbens:F=45.88, p<0.001; n=8).

### Example 2

### Analysis of the Treatment Effect of Combinations of Methylphenidate and Naltrexone on Body Weight in Mice

This example illustrates the treatment of obesity in a mouse model via the administration of a combination of methylphenidate and naltrexone.

### Materials and Methods

C57/B6 mice (male, 4 week old) are purchased from Charles River Laboratories (Kingston, R.I.) and singly housed in standard mouse shoebox cages on a 12 hr. light-dark cycle. Each mouse is provided with an ad libitum supply of drinking water and a high fat rodent diet (45% kCal % diet, pellets, #D12451, Research Diets Inc., New Brunswick, N.J.) for eight weeks.

It is anticipated that the mice nearly double their body weight: for example, from approximately 20 g at the start of the high fat diet to approximately 40 g at the end of an 8-week period.¹⁵

At the end of the 8-week period, the mice are divided into 6 groups as shown is Table 1, wherein each group has 10 mice. Each group receives the drugs or vehicle specified in Table 1, respectively, via daily intra-peritoneal injections.

TABLE 1

Drug (s)

| Mouse group | Vehicle | methylphenidate | naltrexone |
|---|---|---|---|
| A (control) | saline | | |
| B | | 0.75 mg/kg | |
| C | | 1.5 mg/kg | |
| D | | 0.75 mg/kg | 10 mg/kg |
| E | | 1.5 mg/kg | 10 mg/kg |
| F | | | 10 mg/kg |

At the end of the 8-week period of weight gain, and throughout the vehicle/drug treatment period, 2 mice from each group are housed in metabolic chambers to measure locomotor activity, caloric and water intake, and energy expenditure.^{16,17}

For group B and group D, methylphenidate is administered to mice at a dose of 0.75 mg/kg. 0.75 mg/kg methylphenidate is established to reduce hyperactivity in a prenatal nicotine exposure mouse model of ADHD¹⁸ and to result in the same dose of D-methylphenidate (the therapeutically active enantiomer) in the plasma and brain of the mouse as those found in ADHD subjects receiving therapeutic doses of methylphenidate.¹⁹ The dose of methylphenidate at 0.75 mg/kg is believed to be the therapeutic equivalent and the safe dose of methylphenidate in the mouse model. It is predicted that this dose will produce weight loss in the mouse model.

A higher dose (1.5 mg/kg) of methylphenidate for group C is adopted to evaluate dose-response relationships.

As shown in FIG. 4, administered with 7.5 mg/kg methylphenidate (10 times the therapeutic equivalent dose and the dose likely used by abusers), naltrexone at 10 mg/kg completely abolishes the abuse potential of methylphenidate.¹¹ This naltrexone dose of 10 mg/kg is used for Groups D, E and F, respectively. In Group D, naltrexone is administered to the mice at a dose of 10 mg/kg in combination with a therapeutic dose (0.75 mg/kg) of methylphenidate. In Group E, naltrexone is administered to the mice at a dose of 10 mg/kg in combination with a high dose (1.5 mg/kg) of methylphenidate. Naltrexone alone is administered to the mice at a dose of 10 mg/kg for Group F.

Every day for up to 8 weeks, the drugs or vehicle are administered to the mice at 5 PM, two hours prior to the start of the “active” or dark phase of the diurnal cycle (lights off at 7 PM). Each mouse is weighed daily right before the administration of drugs or vehicle.

Since Human subjects are likely to take the anti-obesity drugs during the day when the period is the active phase of human subjects, an active phase of mice is chosen for the testing on mice. Rodents are nocturnal creatures, thus the lights-off period is their active phase when their food consumption increases. In addition, it is discovered that hyperactivity in an ADHD mouse model started to decline 2 hr after the methylphenidate administration.¹¹ In other words, about 2 hr after the administration of methylphenidate, methylphenidate starts to show apparent behavioral effects on mouse model. It is known that an action of stimulants is to suppress food intake, methylphenidate's anti-obesity effects are more likely to be detected if it is administered at the beginning of the active phase.

Statistical Analysis: ANOVA is used for analyzing the data to evaluate the main effects of methylphenidate, naltrexone, and the combination of methylphenidate and naltrexone; and to evaluate the interaction between the two drug treatments.

### Expected Findings

### Weight Loss

It is anticipated that the 8-week period of drug administration is sufficient to produce at least a 10% reduction in weight in the drug treated mice. Weight loss may be proportional to the decreased food intake and/or the dose of methylphenidate administered to the mice. Naltrexone alone may produce weight loss.²⁰ Compared to naltrexone or methylphenidate being used alone, the combination of methylphenidate and naltrexone may produce a greater weight loss than either drug alone. A significant statistical interaction may exist between the effects of methylphenidate alone and naltrexone alone such that the two compounds, when administered together, may be more effective than either compound on its own.

The ascending order of weight loss in the groups may occur as follows: a) Saline (Group A), b) Naltrexone 10 mg/kg (Group F), c) Methylphenidate 0.75 mg/kg (Group B), d) Methylphenidate 1.5 mg/kg (Group C), e) Methylphenidate 0.75 mg/kg+naltrexone 10 mg/kg (Group D), and f) Methylphenidate 1.5 mg/kg+naltrexone 10 mg/kg (Group E)

### Alternative Outcomes

The 8-week period of drug treatment may be too long and the weight gain in the mice in Group A (vehicle treated) may be too high. If this turns out to be the case, the drug treatment earlier is terminated earlier. The data for the body weights are recorded on a daily basis and may be used to measure the rate of weight gain. In case the experiment needed to be terminated before a 10% weight loss is achieved (because of pathologically obese controls), the rate of weight loss may be used to extrapolate the time needed to achieve 10% weight loss.

The maximum weight loss may occur in the initial 2-4 weeks and levels off thereafter.

Now, it is unknown if the weight loss will be sustained even after the termination of the drug treatment.

### Example 3

### Use Utilities

A therapeutically effective amount of a combination of one or more CNS stimulants and/or pharmaceutically acceptable analogs, salts, or hydrates of the CNS stimulants and one or more non-selective opioid receptor antagonists and/or pharmaceutically acceptable analogs, salts, or hydrates of the one or more non-selective opioid receptor antagonists is used for the treatment of obesity.

### Routes of Administration

A therapeutically effective amount of one or more stimulants and one or more non-selective opioid receptor antagonists and/or pharmaceutically acceptable analogs, salts, or hydrates of the one or more non-selective opioid receptor antagonists can be administered in a combination by a variety of routes. In effecting treatment of a subject afflicted with obesity, the composition can be administered in any form or mode that makes the composition bioavailable in an effective amount. The routes encompass oral and parenteral routes. For example, the compounds can be administered orally, by inhalation, or by the subcutaneous, intramuscular, intravenous, transdermal, intranasal, rectal, ocular, topical, sublingual, buccal, or other routes.

One skilled in the art of preparing formulations can readily select the proper form and mode of administration depending upon the particular characteristics of the compound selected, the disorder or condition to be treated, the stage of the disorder or condition, and other relevant circumstances.

### Pharmaceutical Compositions

The pharmaceutical compositions are prepared in a manner well known in the pharmaceutical art, wherein the pharmaceutical compositions comprise therapeutically effective amount of combinations of one or more stimulants and/or pharmaceutically acceptable analogs, salts, or hydrates of the one or more stimulants; and one or more non-selective opioid receptor antagonists and/or pharmaceutically acceptable analogs, salts, or hydrates of the one or more non-selective opioid receptor antagonists. The carrier or excipient may be a solid, semi-solid, or liquid material that can serve as a vehicle or medium for the active ingredient. Suitable carriers or excipients are well known in the art.

The pharmaceutical composition may be adapted for oral or parenteral and may be administered to the subject in the dosage form of tablets, sugar-coated tablets, capsules, delayed-release hard capsules, softgel, chewable tablets, gummy, caplets, powders, granules, syrups, aerosols, inhalants, suppositories, solutions, suspensions, catheters containing the composition, syringes containing the composition, implants containing the composition, transdermal patch, or the like.

A therapeutically effective amount of one or more non-selective opioid receptor antagonists and/or pharmaceutically acceptable analogs, salts, or hydrates thereof may be formulated in an immediate release dosage form; and a therapeutically effective amount of one or more stimulants and/or pharmaceutically acceptable analogs, salts, or hydrates thereof may be formulated in a delayed-release dosage form. After administration, the one or more stimulants and/or pharmaceutically acceptable analogs, salts, or hydrated thereof may be released 30 minutes later than the release of the one or more non-selective opioid receptor antagonists and/or pharmaceutically acceptable analogs, salts, or hydrated thereof. For example, a fix-combination comprising naltrexone and MPH in a therapeutically effect amount may be formulated in a dosage form, wherein after administration to a subject, naltrexone releases earlier than the release of MPH.

In some examples, a combination may be a fixed-dose combination, wherein the one or more stimulants and/or pharmaceutically acceptable analogs, salts or hydrates thereof and the one or more non-selective opioid receptor antagonists and/or pharmaceutically acceptable analogs, salts or hydrates thereof are combined in one preparation, in one dosage form, or in a single entity of dosage such as a tablet, a softgel, a hard capsule, a chewable tablet, a gummy, a polypill, a transdermal patch, liquid, a syrup, etc. All compounds are intermixed in a same pharmaceutically acceptable carrier.

In some other examples, a combination may be a non-fixed combination, wherein the one or more CNS stimulants and/or pharmaceutically acceptable analogs, salts or hydrates thereof and the one or more non-selective opioid receptor antagonists and/or pharmaceutically acceptable analogs, salts or hydrates thereof exist in separate dosage forms or as separate entities. Compositions in separate dosage forms may be mixed together before an administration to a subject having a need thereof.

The dosage form for the one or more non-selective opioid receptor antagonists and/or pharmaceutically acceptable analogs, salts, or hydrated thereof may be different from for the one or more stimulants and/or pharmaceutically acceptable analogs, salts, or hydrated thereof.

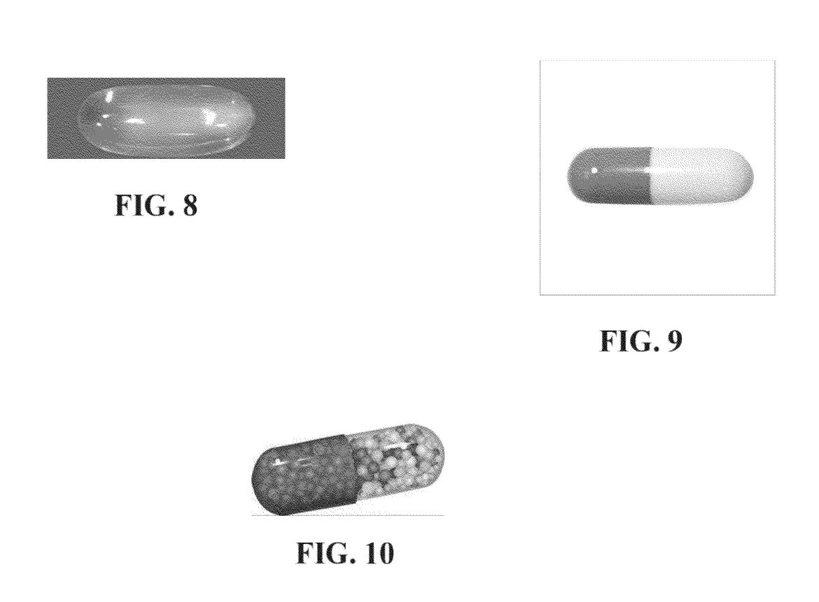

In one example, a combination disclosed herein may be formulated in a dosage form of a softgel as shown in FIG. 8. A combination disclosed herein may be formulated in a dosage form of hard capsule as shown in FIG. 9. A combination disclosed herein may also be formulated in a dosage form of a hard capsule wherein each different compound in the combination is coated differently (FIG. 10). For example, in a hard capsule as shown in FIG. 10, one or more CNS stimulants and/or pharmaceutically acceptable analogs, salts, or hydrates of the one or more CNS stimulants may be in controlled release coatings, while one or more non-selective opioid receptor antagonists and/or pharmaceutically acceptable analogs, salts, or hydrates of the one or more non-selective opioid receptor antagonists may be in immediate release or fast release coatings. The different coatings for different compounds in the combination allow different compounds to be released at different period time as desired. For example, in a hard capsule comprising a therapeutically effective amount of naltrexone and a therapeutically effective amount of MPH, naltrexone may be prepared in a coating that allow the immediate or fast release of naltrexone after administration, and MPH may be prepared in a coating that delays the release of MPH. The period between the release of naltrexone and the release of MPH may be at least more than about 30 minutes.

In an example, a capsule can be designed in which an insoluble core of naltrexone is encased within a soluble shell of MPH so that when the capsule is taken orally, as prescribed, only the MPH is absorbed and the naltrexone core passes through the gastro-intestinal tract intact. If the capsule is crushed (for abuse), the naltrexone is released and would mitigate the abuse potential of MPH. Such preparation may alleviate concerns about potential side effects of high doses of naltrexone, including actions at κ and δ opioid receptors, as well as naltrexone's potential interaction with therapeutic action of MPH.

A capsule, in an example, can be designed in which an insoluble core of naltrexone is encased within a soluble shell of MPH so that when the capsule is taken orally, as prescribed, only the MPH is absorbed and the naltrexone core passes through the gastro-intestinal tract intact. If the capsule is crushed (for abuse), the naltrexone is released and would mitigate the abuse potential of MPH. Such preparation may alleviate concerns about potential side effects of high doses of naltrexone, including actions at κ and δ opioid receptors, as well as naltrexone's potential interaction with therapeutic action of MPH.

The above design is not limited to naltrexone and MPH. Other compounds of the one or more non-selective opioid receptor antagonists and/or pharmaceutically acceptable analogs, salts, or hydrated thereof may be formulated into an insoluble core of a capsule and other compounds of the one or more CNS stimulants and/or pharmaceutically acceptable analogs, salts, or hydrated thereof may be formulated into a soluble shell of the capsule.

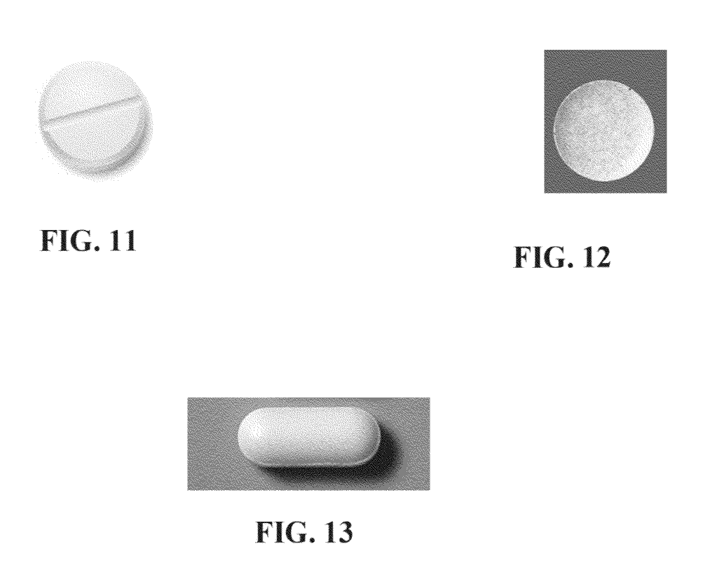

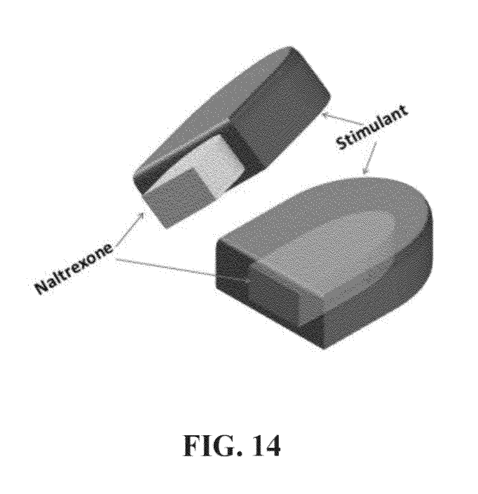

In another example, a combination disclosed herein may be formulated in a dosage form of a tablet as shown in FIG. 11. A tablet comprising a combination disclosed herein may be a chewable tablet as shown in FIG. 12. A combination disclosed herein may also be formulated in a caplet as shown in FIG. 13. A caplet comprising a combination disclosed herein may be formulated as a controlled-release caplet comprising two or more portions, such as a core and a shell, wherein the core is within the shell, as shown in FIG. 14. Each portion in such controlled-release caplet may comprise different compounds in the combination. For example, a core in such controlled-release caplet may comprise naltrexone and the shell may comprise MPH. The controlled-release caplet may be designed as that when the controlled-release caplet is taken orally, as prescribed, naltrexone comprised in the core is released immediately and MPH comprised in the shell is delayed-released. The period between the release of naltrexone and the release of MPH may be at least more than about 30 minutes.

The above examples are not limited to combinations comprising naltrexone and MPH. The provided dosage forms also apply to combinations comprising any one or more non-selective opioid receptor antagonists and/or pharmaceutically acceptable analogs, salts, or hydrated thereof and any one or more CNS stimulants and/or pharmaceutically acceptable analogs, salts, or hydrated thereof in therapeutically effective amount.

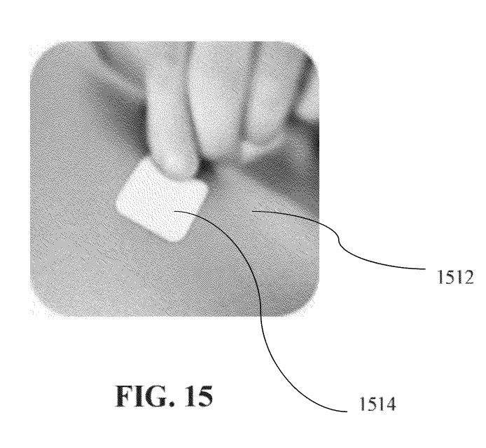

In addition to above examples of dosage forms for orally administration, in some examples, a combination disclosed herein may be delivered into a subject's body via other approaches such as via a transdermal patch. FIG. 15 illustrates a transdermal patch 1514 comprising a combination disclosed herein. Transdermal patch 1514 may comprise an adhesive patch for placing on the skin 1512 of a subject and one or more active layers embedded in the adhesive patch, wherein the one or more active layers comprise a combination disclosed herein in a therapeutically effective amount. Transdermal patch 1614 may be placed on to a subject's body to allow the combination embedded in the adhesive patch be delivered into the subject's bloodstream.

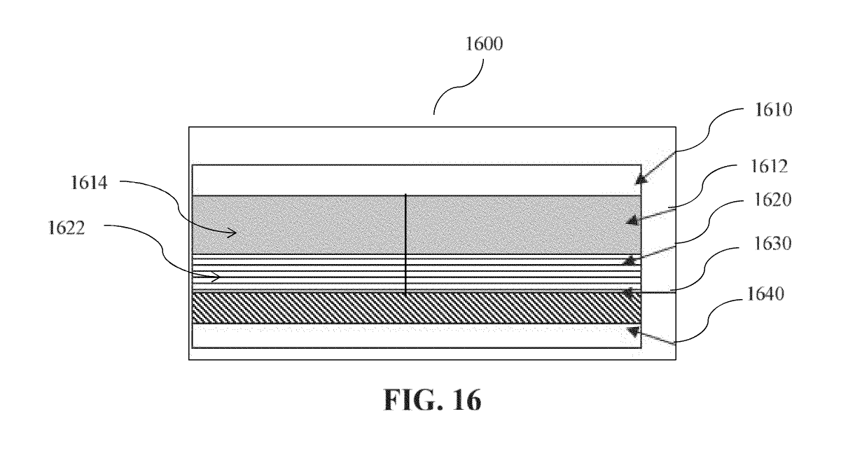

In some examples, as shown in FIG. 16, in a transdermal patch 1600, a combination of a therapeutically effective amount of naltrexone and a therapeutically effective amount of MPH are formulated into one or more active layers (for example, 1612 and 1614) and embedded under an impermeable backing layer 1610. For example, a combination comprises naltrexone and MPH in therapeutically effective amount, wherein naltrexone is formulated into an active layer 1612 and MPH is formulated into an active layer 1614. The active layer comprising naltrexone 1612 is further attached to a first rate controlling membrane 1620 and the active layer 1614 comprising MPH is further attached to a second rate controlling membrane 1622. The first rate controlling membrane 1620 and the second controlling membrane 1622 are further attached to an adhesive layer 1630, which adhesive layer 1630 further attaches to a release liner 1640. Under the control of the first rate controlling membrane 1620 and the second rate controlling membrane 1622, respectively, the release of naltrexone is at a different rate from that of the release of MPH. The above example is not limited to a combination that comprises a therapeutically amount of naltrexone and MPH; it may also apply to a combination comprising one or more CNS stimulants and/or pharmaceutically acceptable analogs, salts, or hydrates of the one or more CNS stimulants and one or more non-selective opioid receptor antagonists and/or pharmaceutically acceptable analogs, salts, or hydrates of the one or more non-selective opioid receptor antagonists in a therapeutically effective amount.

FIG. 15 and FIG. 16 are only illustrative representations of an exemplary delivery of a disclosed combination into a subject's body through a transdermal patch. One of ordinary skill in the art would readily appreciate that any kind of transdermal patch suitable for delivering the disclosed products described in the present invention may be utilized.

### Nutraceutical Compositions

The combination of a therapeutically effective amount of naltrexone and/or pharmaceutically acceptable analogs, salts, or hydrates of naltrexone and a therapeutically effective amount of methylphenidate and/or pharmaceutically acceptable analogs, salts, or hydrates of methylphenidate may be formulated into a nutraceutical composition.

The term nutraceutical as used herein denotes the usefulness in both the nutritional and pharmaceutical field of application. Thus, the nutraceutical compositions can find use as supplement to food and beverages, and as pharmaceutical formulations for enteral or parenteral application which may be solid formulations such as capsules or tablets, or liquid formulations, such as solutions or suspensions. Nutraceutical compositions may also comprise food and beverages containing therapeutically effective amount of one or more non-selective opioid receptor antagonists, CNS stimulants, pharmaceutically acceptable analogs, salts or hydrates of the one or more respective non-selective opioid receptor antagonists, CNS stimulants as well as supplement compositions, for example dietary supplements. For example, a nutraceutical supplement may comprise naltrexone and MPH in a therapeutically effective amount.

### Doses

The compounds may be used at appropriate dosages defined by routine testing to obtain optimal pharmacological effect, while minimizing any potential toxic or otherwise unwanted effects.

An effective amount can be readily determined by the attending diagnostician, as one skilled in the art, by the use of conventional techniques and by observing results obtained under analogous circumstances. In determining an effective amount, the dose of a compound, a number of factors are considered by the attending diagnostician, including, but not limited to: the compound to be administered; the species of mammal—its size, age, and general health; the specific disorder involved; the degree of involvement or the severity of the disorder; the response of the individual subject; the mode of administration; the bioavailability characteristics of the preparation administered; the dose regimen selected; the use of other concomitant medication; and other relevant circumstances.

The specific dose administered may be determined by particular circumstances surrounding each situation. These circumstances can include: the route of administration, the prior medical history of the recipient, the symptom being treated, the severity of the symptom being treated, and the age of the recipient. The recipient subject's attending physician should determine the therapeutic dose administered in light of the relevant circumstances.

Also, it is to be understood that the exact dose may be determined, in accordance with the standard practice in the medical arts of “dose titrating” the recipient; that is, initially administering a low dose of the compound, and gradually increasing the dose until the desired therapeutic effect is observed.

It is to be further understood that the dosage regimen can be selected in accordance with a variety of factors. These include type, species, age, weight, sex, diet, and medical condition of the subject; the severity of the condition to be treated; the route of administration; the kidney and liver functions of the subject; the time of administration; the rate of excretion; and the particular compounds employed. A physician of ordinary skill can readily determine and prescribe the effective amount of the drug required to prevent, counter or arrest the progress of the disease or disorder that is being treated.

For example, naltrexone may be administered to a human subject at a daily dose from about 0.5 mg/kg to about 1.5 mg/kg of the human subject's body weight, and MPH may be administered to a human subject at a daily dose from about 0.75 mg/kg to about 2 mg/kg of the human subject's body weight. The amount of naltrexone and MPH in a combination may be flexible according to need. For example, in one embodiment, a daily dose of combination may comprise about 0.5 mg/kg of naltrexone and about 0.75 mg/kg of MPH. In another example, a daily dose of combination may comprise about 1.0 mg/kg naltrexone and about 1.0 mg/kg of MPH. The examples are not exclusive. The therapeutically effective amount of MPH and naltrexone in a combination administered to a human subject per day may be any combination of about 0.75 to about 2 mg/kg of MPH and about 0.5 to 1.5 mg/kg of naltrexone. The daily amount of MPH in a combination may be less than about 0.75 mg/kg or more than 2 mg/kg, and the daily amount of naltrexone may be less than 0.5 mg/kg or more than 1.5 mg/kg, depending on the need for providing optimal therapeutic responses.

In one example, this product may be used for administration according to a continuous schedule having a dosing interval selected from one or more of: once daily dosing and/or multiple daily dosing. In one embodiment, this product may be administered to a subject having a need thereof chronically.

Although a combination of methylphenidate and naltrexone is an example for treating obesity, it will be appreciated that other members of the class of non-selective opioid receptor antagonist and other members in the class of stimulants can be used for the treatment of obesity. The subject receiving such treatment is not limited to mice. Obesity is not limited to be induced by high fat diet. It could be any reason, including those unknown.

It is intended that the invention not be limited to the particular embodiment disclosed herein contemplated for carrying out this invention, but that the invention will include all embodiments falling within the scope of the claims.

All documents, patents, journal articles and other materials cited in the present application are incorporated herein by reference.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

### REFERENCES

The following references are referred to above and are incorporated herein by reference:
1. Adan R A (2013) Mechanisms underlying current and future anti-obesity drugs. Trends in Neurosciences.
2. Bray G A (1994) Amphetamine: the Janus or treatment for obesity. Obesity Research 2 282-265.
3. Colman E (2012) Food and Drug Administration's Obesity Drug Guidance Document: a short history. Circulation 125:2156-2164
4. Brown R T, Amler R W, Freeman W S. Perrin J M, Steir M T, Feldman H M, Pierce K, Wolraich M L (2005) Treatment of attention-deficit/hyperactivity disorder overview of the evidence. Pediatrics 115: e749-757.
5. Biederman J, Arnsten A F, Faraone S V, Doyle A E, Spencer T J, Wilens T E, Weiss M D, Safren S A, Culpepper L (2006) New developments in the treatment of ADHD. J Clin Psychiatry 67:148.159
6. Klein-Schwartz W (2002) Abuse and toxicityi methylphenldale. Curr Opin Pediatr 14:219-223
7. Teter C J, McCabe S E, Boyd C J. Guthrie S K (2003) Illicit methylphenidate use in an undergraduate student sample: prevalence and risk factors. Pharmacotherapy 23: 609-617
8. McCabe S E, Teter C J, Boyd C J, Guthrie S K (2004) Prevalence and correlates of illicit methylphenidate use among 8th, 10th, and 12th grade students in the United States, 2001. J Adolesc Health 35:501-504
9. Bright G M (2008) Abuse of medications employed for the treatment of ADHD: results from a large-scale community survey. Medscape J Med 10:111
10. New York Times, Jun. 19, 2012.
11. Zhu J, Spencer T J, Liu-Chen L Y, Biederman J, Bhide P G (2011) Methylphenidate and mu opioid receptor interactions: a pharmacological target for prevention of stimulant abuse. Neuropharmacology 61:283-292
12. Liang N C, Bello N T, Moran T H (2013) Additive feeding inhibitory and aversive effects of naltrexone and exendin-4 combinations. Int J Obes 37:272-278
13. Rueda-Clausen C F, Padwal R S, Sharma A M (2013) New pharmacological approaches for obesity management. Nat Rev Endocrinol 9:467-478
14. Tek C, Guloksuz, S, Srihari V H, Reutenauer E L (2013) Investigating the safety and efficacy of naltrexone for anti-psychotic induced weight gain in severe mental illness: study protocol of a double-blind, randomized, placebo-controlled trial. BMC Psychiatry 13:176.
15. Van Heek M, Compton D S, France C F, Tedesco R P, Fawzi A B, Graziano M P, Sybertz E J, Strader C D, Davis H R, Jr. (1997) Diet-induced obese mice develop peripheral, but not central, resistance to leptin. The Journal of clinical investigation 99:385-390.
16. Tucker K, Overton J M, Fadool D A (2008) Kv1.3 gene-targeted deletion alters longevity and reduces adiposity by increasing locomotion and metabolism in melanocortin-4 receptor-null mice. Int J Obes 32:1222-1232.
17. Tucker K, Overton J M, Fadool D A (2012) Diet-induced obesity resistance of Kv1.3−/− mice is olfactory bulb dependent. Journal of neuroendocrinology 24:1087-1095.
18. Zhu J, Zhang X, Xu Y, Spencer T J, Biederman J, Bhide P G (2012) Prenatal nicotine exposure mouse model showing hyperactivity, reduced cingulate cortex volume, reduced dopamine turnover, and responsiveness to oral methylphenidate treatment. The Journal of neuroscience: the official journal of the Society for Neuroscience 32:9410-9418.
19. Balcioglu A, Ren J Q, McCarthy D, Spencer T J, Biederman J, Bhide P G (2009) Plasma and brain concentrations of oral therapeutic doses of methylphenidate and their impact on brain monoamine content in mice. Neuropharmacology 57:687-693.
20. Giuliano C, Robbins T W, Nathan P J, Bullmore E T, Everitt B J (2012) Inhibition of opioid transmission at the mu-opioid receptor prevents both food seeking and binge-like eating. Neuropsychopharmacology: official publication of the American College of Neuropsychopharmacology 37:2643-2652.

What is claimed is:

1. A method for treating obesity comprising administering to a subject having a need thereof a combination comprising two or more compounds, wherein the two or more compounds comprise:
a therapeutically effective amount of naltrexone and/or pharmaceutically acceptable analogs, salts, or hydrates of naltrexone; and
a therapeutically effective amount of methylphenidate and/or pharmaceutically acceptable analogs, salts, or hydrates of methylphenidate.

2. The method of claim 1, wherein methylphenidate and/or pharmaceutically acceptable analogs, salts or hydrates of methylphenidate are administered to a human subject at a daily dose from about 0.75 mg/kg to about 2 mg/kg of subject body weight.

3. The method of claim 1, wherein naltrexone and/or pharmaceutically acceptable analogs, salts or hydrates of naltrexone are administered to a human subject at a daily dose from about 0.5 mg/kg to about 1.5 mg/kg of subject body weight.

4. The method of claim 1, wherein the two or more compounds in the combination are simultaneously administered to the subject.

5. The method of claim 1, wherein the two or more compounds in the combination are administered to the subject sequentially; and wherein the naltrexone and/or pharmaceutically acceptable analogs, salts, or hydrates of naltrexone are administered to the subject prior to an administration of methylphenidate and/or pharmaceutically acceptable analogs, salts, or hydrates of methylphenidate.

6. The method of claim 1, wherein the two or more compounds in the combination are administered to the subject sequentially; and wherein the naltrexone and/or pharmaceutically acceptable analogs, salts, or hydrates of naltrexone is administered to the subject about 30 minutes prior to an administration of methylphenidate and/or pharmaceutically acceptable analogs, salts, or hydrates of methylphenidate.

7. The method of claim 1, wherein the combination is orally administered to the subject.

8. The method of claim 1, wherein the combination is administered to the subject via parenteral route.

9. The method of claim 1, wherein the combination is administered to the subject via intranasal route.

10. The method of claim 1, wherein the combination is administered to the subject with a pharmaceutical carrier.

11. The method of claim 1, wherein the combination is administered to the subject with a nutraceutical carrier.

12. The method of claim 1, wherein the combination is chronically administered to the subject.

13. The method of claim 1, wherein the subject has, or is at risk of developing, a food addiction.

14. The method of claim 1, wherein the subject has, or is at risk of developing, a binge-eating disorder, or engages in a binge eating behavior.

15. A method for treating obesity comprising administering to a subject having a need thereof a combination comprising two or more compounds, wherein the two or more compounds comprise:
a therapeutically effective amount of one or more non-selective opioid receptor antagonists and/or pharmaceutically acceptable analogs, salts, or hydrates of the one or more non-selective opioid receptor antagonists; and
a therapeutically effective amount of one or more CNS stimulants and/or pharmaceutically acceptable analogs, salts, or hydrates of the one or more CNS stimulants.

16. The method of claim 15, wherein the one or more CNS stimulants comprise methylphenidate.

17. The method of claim 15, wherein the one or more CNS stimulants comprise amphetamine.

18. The method of claim 15, wherein the one or more CNS stimulants comprise one or more stimulant-like compounds.

19. The method of claim 15, wherein the one or more CNS stimulants comprise analeptic.

20. The method of claim 15, wherein the one or more non-selective opioid antagonists comprise naltrexone.

21. The method of claim 15, wherein the two or more compounds comprised in the combination are administered to the subject simultaneously.

22. The method of claim 15, wherein the two or more compounds comprised in the combination are administered to the subject separately.

23. The method of claim 15, wherein the two or more compounds comprised in the combination are sequentially administered to the subject, wherein the one or more non-selective opioid receptor antagonists and/or pharmaceutically acceptable analogs, salts, or hydrates of the one or more non-selective opioid receptor antagonists is administered prior to an administration of the one or more CNS stimulants and/or pharmaceutically acceptable analogs, salts, or hydrates of the one or more CNS stimulants.

24. The method of claim 15, wherein the two or more compounds comprised in the combination are administered to the subject sequentially, wherein the one or more non-selective opioid receptor antagonists and/or pharmaceutically acceptable analogs, salts or hydrates of the one or more non-selective opioid receptor antagonists is administered about 30 minutes prior to an administration of the one or more CNS stimulants and/or pharmaceutically acceptable analogs, salts, or hydrates of the one or more CNS stimulants.

25. The method of claim 15, wherein the combination is orally administered to the subject.

26. The method of claim 15, wherein the combination is administered to the subject via parenteral route.

27. The method of claim 15, wherein the combination is administered to the subject via intranasal route.

28. The method of claim 15, wherein the combination is administered to the subject with a pharmaceutical carrier.

29. The method of claim 15, wherein the combination is administered to the subject with a nutraceutical carrier.

30. The method of claim 15, wherein the subject has, or is at risk of developing, a food addiction.

31. The method of claim 15, wherein the subject has, or is at risk of developing, a binge-eating disorder, or engages in a binge eating behavior.

32. A product comprising at least one dosage of a combination for treating obesity, wherein the combination comprises two or more compounds, and wherein the two or more compounds comprise:
a therapeutically effective amount of naltrexone and/or pharmaceutically acceptable analogs, salts, or hydrates of naltrexone; and
a therapeutically effective amount of methylphenidate and/or pharmaceutically acceptable analogs, salts, or hydrates of methylphenidate.

33. The product of claim 32, wherein the product comprises a nutraceutical composition for oral administration.

34. The product of claim 32, wherein the product comprises a pharmaceutical composition.

35. The product of claim 32, wherein the product comprises a fixed-dose combination.

36. The product of claim 35, wherein the product comprises a tablet comprising the combination.

37. The product of claim 35, wherein the product comprises a softgel comprising the combination.

38. The product of claim 35, wherein the product comprises a chewable tablet comprising the combination.

39. The product of claim 35, wherein the product comprises a syrup comprising the combination.

40. The product of claim 35, wherein the product comprises a capsule comprising the combination.

41. The product of claim 40, wherein the capsule comprises an insoluble core encased within a soluble shell,
wherein the insoluble core comprises naltrexone and/or pharmaceutically acceptable analogs, salts, or hydrates of naltrexone,
wherein the soluble shell comprises MPH and/or pharmaceutically acceptable analogs, salts, or hydrates of MPH, and
wherein when the capsule is taken orally, only MPH and/or pharmaceutically acceptable analogs, salts, or hydrates of MPH are absorbed and the insoluble core passes through gastro-intestinal tract intact.

42. The product of claim 32, wherein the naltrexone and/or pharmaceutically acceptable analogs, salts, or hydrates of naltrexone are in an immediate release dosage form, and methylphenidate and/or pharmaceutically acceptable analogs, salts, or hydrates of methylphenidate are in a delayed-release dosage form.

43. The product of claim 32, wherein the therapeutically effective amount of naltrexone and/or pharmaceutically acceptable analogs, salts, or hydrates of naltrexone and the therapeutically effective amount of methylphenidate and/or pharmaceutically acceptable analogs, salts, or hydrates of methylphenidate are in separate dosage forms, and wherein the separate dosage forms can be mixed together before an administration of the combination to a subject having a need thereof.

44. A product comprising at least one dosage of a combination for treating obesity, wherein the combination comprises a therapeutically effective amount of two or more compounds, and wherein the two or more compounds comprise:
one or more non-selective opioid receptor antagonists and/or pharmaceutically acceptable analogs, salts or hydrates of the one or more non-selective opioid receptor antagonists; and
one or more CNS stimulants and/or pharmaceutically acceptable analogs, salts, or hydrates of the one or more CNS stimulants.

45. The product of claim 44, wherein the one or more CNS stimulants comprise methylphenidate.

46. The product of claim 44, wherein the one or more CNS stimulants comprise amphetamine.

47. The product of claim 44, wherein the one or more CNS stimulants comprise one or more stimulant-like compounds.

48. The product of claim 44, wherein the one or more CNS stimulants comprise analeptic.

49. The product of claim 44, wherein the one or more non-selective opioid antagonists comprise naltrexone.

50. The product of claim 44, wherein the product comprises an orally administrable nutraceutical composition and wherein the nutraceutical composition comprises the two or more compounds.

51. The product of claim 44, wherein the product comprises a pharmaceutical composition comprising the two or more compounds.

52. The product of claim 44, wherein the product comprises a fixed-dose combination

53. The product of claim 52, wherein the product comprises a tablet comprising the combination.

54. The product of claim 52, wherein the product comprises a softgel comprising the combination.

55. The product of claim 52, wherein the product comprises a caplet comprising the combination.

56. The product of claim 52, wherein the product comprises an injection fluid comprising the combination.

57. The product of claim 52, wherein the product comprises a chewable tablet comprising the combination.

58. The product of claim 52, wherein the product comprises a syrup comprising the combination.

59. The product of claim 52, wherein the product comprises a capsule comprising the combination.

60. The product of claim 59, wherein the capsule comprises an insoluble core encased within a soluble shell;
wherein the insoluble core comprises one or more non-selective opioid receptor antagonists and/or pharmaceutically acceptable analogs, salts or hydrates of the one or more non-selective opioid receptors;
wherein the soluble shell comprises one or more CNS stimulants and/or pharmaceutically acceptable analogs, salts, or hydrates of the one or more CNS stimulants; and
wherein when the capsule is taken orally, only the one or more CNS stimulants and/or pharmaceutically acceptable analogs, salts, or hydrates of the one or more CNS stimulants are absorbed and the insoluble core passes through gastro-intestinal tract intact.

61. The product of claim 44, wherein the one or more non-selective opioid receptor antagonists and/or pharmaceutically acceptable analogs, salts or hydrates of the one or more non-selective opioid receptors are in an immediate release dosage form; and the one or more CNS stimulants and/or pharmaceutically acceptable analogs, salts, or hydrates of the one or more CNS stimulants are in a delayed-release dosage form.

62. The product of claim 44, wherein the two or more compounds are in separate dosage forms, and wherein the separate dosage forms can be mixed together before an administration of the combination to a subject having a need thereof.

63. A treatment delivery apparatus comprising a treatment carrier device and at least one dosage of a combination contained in the treatment carrier device for treating obesity, wherein the combination comprises:
a therapeutically effective amount of one or more non-selective opioid receptor antagonists and/or pharmaceutically acceptable analogs, salts or hydrates of the one or more non-selective opioid receptor antagonists; and
a therapeutically effective amount of one or more CNS stimulants and/or pharmaceutically acceptable analogs, salts, or hydrates of the one or more stimulants.

64. The treatment delivery apparatus of claim 63, wherein the one or more non-selective opioid receptor antagonists comprise naltrexone, and wherein the one or more CNS stimulants comprise methylphenidate.

65. A treatment delivery apparatus comprising a transdermal patch,
wherein the transdermal patch comprises an adhesive patch for placing the transdermal patch on a skin of a subject and one or more active layers embedded in the adhesive patch,
wherein the one or more active layers comprise a combination comprising a therapeutically effective amount of two or more compounds for treating obesity, and
wherein the two or more compounds comprise:
one or more non-selective opioid receptor antagonists and/or pharmaceutically acceptable analogs, salts or hydrates of the one or more non-selective opioid receptor antagonists; and
one or more CNS stimulants and/or pharmaceutically acceptable analogs, salts, or hydrates of the one or more CNS stimulants.

66. The treatment delivery apparatus of claim 65, wherein the one or more non-selective opioid receptor antagonists comprise naltrexone, and wherein the one or more CNS stimulants comprise methylphenidate.
